(19) United States
(12) Patent Application Publication
(10) Pub. No.: US 2019/0321496 A1
(43) Pub. Date: Oct. 24, 2019

(54) AGENTS AND METHODS FOR THE DIAGNOSIS AND TREATMENT OF DISEASES ASSOCIATED WITH EXTRACELLULAR MATRIX TURNOVER

(71) Applicant: Baker IDI Heart and Diabetes Institute Holdings Limited, Melbourne (AU)

(72) Inventors: Christoph Eugen Hagemeyer, Cheltenham (AU); Mei Yee Choy, Dunman View (SG)

(21) Appl. No.: 16/310,282

(22) PCT Filed: Jun. 16, 2017
(86) PCT No.: PCT/AU2017/050605

§ 371 Date: Dec. 14, 2018

(30) Foreign Application Priority Data
Jun. 16, 2016 (AU) 2016902338

(51) Int. Cl. A61K 51/08; A61P 9/10; A61K 47/69; A61K 47/65; A61K 47/62; A61K 31/365

(52) U.S. Cl. CPC A61K 51/088; A61P 9/10; A61K 31/365; A61K 47/65; A61K 47/62; A61K 47/6929

(57) ABSTRACT

The present disclosure relates generally to agents that target areas of extracellular matrix turnover in biological tissue and their use for the diagnosis or treatment of conditions associated with extracellular matrix turnover, wherein the agents comprise a polypeptide comprising the amino acid sequence TLTYTWS (SEQ ID NO:1).

### FIELD OF THE DISCLOSURE

The present disclosure relates generally to agents that target areas of extracellular matrix turnover in biological tissue and their use for the diagnosis or treatment of conditions associated with extracellular matrix turnover, such as atherosclerosis and fibrosis.

### BACKGROUND OF THE DISCLOSURE

Atherosclerosis is characterised by the formation of atherosclerotic plaques within the vascular lumen. While most plaques remain quiescent for years, some become unstable, leading to plaque rupture or erosion. The rupture of unstable plaques is currently one of the leading causes of mortality and morbidity worldwide. With increasing rates of obesity and diabetes and the Western life-style adaptation in highly populated countries like China, India and Indonesia, the number of patients with atherosclerosis and associated medical problem is rising substantially. Two of the major complications of atherosclerosis are myocardial infarction (MI) and stroke. Besides being major causes of direct death (˜20% of all deaths are caused by MI in Australia), the consequences of patients who survive MI or stroke are often devastating with massive loss of quality of life and disability. The economic cost of atherosclerosis-related problems to our health care system is also enormous.

Collagen makes up about 60% of the fibrous cap of an atherosclerotic plaque and is an important determinant of structural stability. Numerous studies investigating unstable plaques have demonstrated a direct relationship between fibrous cap thickness, collagen content and the risks of rupture. A cap thickness of less than 65 μm and a decrease in collagen content has been taken to indicate instability and high risk of rupture.

Thus, a key to preventing the complications arising from atherosclerosis, such as MI and stroke, is the identification of unstable, vulnerable atherosclerotic plaques that are prone to rupture. Various invasive, catheter-based methods have been developed with the aim of identifying unstable atherosclerotic plaques, of which intravascular ultrasound (IVUS), optical coherence tomography (OCT) and near-infra-red spectroscopy (NIRS) are the most prominent. However, these methods typically detect only some of the features of vulnerable plaques. Despite an undisputable medical need, enormous commercial potential and already substantial investment from medical device companies, including large clinical trials, there is currently still no technology available that allows for reliable detection and discrimination of unstable atherosclerotic plaques in vivo.

Whilst non-invasive imaging methods such as computed tomography (CT), intravascular ultrasound (IVUS), optical coherence tomography (OCT) and near-infrared spectroscopy (NIRS) are capable of detecting some attributes of atherosclerotic plaques, none of these are capable of reliably identifying unstable, rupture-prone plaques and discriminate these from stable plaques. Coronary angiography has been the gold standard in assessing coronary artery disease, including atherosclerosis. However, coronary angiography only provides information about the vessel lumen; namely, whether or not there is vascular restriction (stenosis). However, vulnerable plaques that develop in the vascular lumen do not necessarily result in stenosis. Indeed, very often the plaques causing stenosis are the ones that are stable and are not prone to rupture. Thus, coronary angiography is not well suited for identifying unstable, vulnerable atherosclerotic plaques. Thus, there still remains an urgent need for a method of identifying unstable, rupture-prone atherosclerotic plaques and discriminating these from stable plaques.

Much as unstable atherosclerotic plaques are characterised by insufficient extracellular matrix deposition and/or turnover, tissue fibrosis in conditions such as chronic heart failure, chronic renal failure and chronic obstructive pulmonary is also characterised by adverse extracellular matrix deposition and/or turnover, resulting in fibroblast accumulation and excess deposition of extracellular matrix proteins that lead to distorted organ architecture and function.

Current techniques for the in vivo imaging of tissue fibrosis have significant limitations. For instance, current techniques for imaging cardiac fibrosis are MRI-based and include late gadolinium enhancement (LGE) of the atrial wall and T1 mapping in the setting of AF and HF. Whilst the former allows spatial identification of fibrosis and correlates well with voltage mapping, it is limited by image quality, uncertain reproducibility and a high degree of operator dependence, largely due to the thinness and hence limited resolution of the atrial wall. T1 mapping has been correlated with atrial scar detected by voltage mapping and to clinical outcomes following AF ablation, however, it is limited in spatial resolution—requiring a regional assessment of atrial areas and has yet to be validated histologically. Similarly, the detection of diffuse ventricular fibrosis by LGE of myocardium, whilst increasingly utilised clinically and has demonstrated an association with adverse outcomes, is fraught with significant limitations including no ability to detect diffuse fibrosis, a lack of standardisation of imaging protocols, and reliance upon an arbitrary scale of signal intensity which may differ from one study to another.

Thus, there remains an urgent need for better agents and methods for the diagnosis and prognosis of conditions associated with unwanted extracellular matrix turnover and fibrosis.

### SUMMARY OF THE DISCLOSURE

The present disclosure is predicated on the inventors' surprising findings that the polypeptide TLTYTWS (SEQ ID NO:1) can differentially bind to unstable atherosclerotic plaques and areas of collagen turnover in vivo. The inventors have also surprisingly shown that this polypeptide can be used as a targeting, moiety in an imaging agent to identify unstable atherosclerotic plaques and areas of collagen turnover (fibrosis) in vivo, as well as a targeting moiety of a therapeutic construct for delivering a therapeutic agent to unstable atherosclerotic plaques and areas of fibrosis in vivo.

Accordingly, in one aspect of the present disclosure, there is provided an imaging agent comprising a polypeptide linked to a detectable label, wherein the polypeptide comprises the amino acid sequence TLTYTWS (SEQ ID NO:1).

In an embodiment disclosed herein, the polypeptide consists of the amino acid sequence TLTYTWS (SEQ ID NO:1). In an embodiment disclosed herein, the detectable label is attached to a complexing agent. In an embodiment disclosed herein, the complexing agent is 1,4,7,10-tetraazacyclododecane-1,4,7,10-tetraacetic acid (DOTA) or 5-(8-methyl-3,6,10,13,16,19-hexaaza-bicyclo[6.6. 6] icosan-1-ylamino)-5-oxopentanoic acid (MeCOSar). In an embodiment disclosed herein, the complexing agent is MeCOSar. In an embodiment disclosed herein, the detectable label is selected from the group consisting of a radio-isotope, an imaging dye, a paramagnetic material or a microbubble. In an embodiment disclosed herein, the detectable label is a radio-isotope. In an embodiment disclosed herein, the radio-isotope is ⁶⁴Cu.

In another aspect of the present disclosure, there is provided a method of detecting an unstable atherosclerotic plaque in a vascular lumen of a subject in vivo, the method comprising: a) administering to a subject in need thereof an imaging agent as herein described; and b) detecting the detectable label of the imaging agent, wherein the presence of the detectable label within the vascular lumen of the subject is indicative of binding of the imaging agent to an unstable atherosclerotic plaque.

In another aspect of the present disclosure, there is provided a method of detecting fibrosis in a subject in vivo, the method comprising: a) administering to a subject in need thereof an imaging agent as herein described; and b) detecting the detectable label of the imaging agent, wherein the presence of the detectable label in the subject is indicative of binding of the imaging agent to an area of fibrosis.

In an embodiment disclosed herein, the method for detecting the detectable label is selected from the group consisting of: single photon emission computed tomography; positron emission tomography; near infrared fluorescence imaging; d) ultrasound imaging; and magnetic resonance imaging. In an embodiment disclosed herein, the method for detecting the detectable label is positron emission tomography (PET). In an embodiment disclosed herein, the subject is a human. In another aspect of the present disclosure, thee is provided a methods for detecting unstable atherosclerotic plaques and/or fibrosis in a biological tissue sample ex vivo, the method comprising: a) contacting a biological tissue sample obtained from a subject with an imaging agent as herein described; and b) detecting the detectable label of the imaging agent, wherein the presence of the detectable label is indicative of binding of the imaging agent to an unstable atherosclerotic plaques and/or an area of fibrosis in the tissue sample.

In another aspect of the present disclosure, there is provided a therapeutic construct comprising a therapeutic moiety linked to a targeting moiety, wherein the targeting moiety comprises a polypeptide comprising the amino acid sequence TLTYTWS (SEQ ID NO:1).

In an embodiment disclosed herein, the polypeptide consists of the amino acid sequence TLTYTWS (SEQ ID NO:1). In an embodiment disclosed herein, the targeting moiety further comprises a cell penetrating agent. In an embodiment disclosed herein, the cell penetrating agent comprises a plurality of positively-charged amino acid residues. In an embodiment disclosed herein, the cell penetrating agent comprises a plurality of D-arginine residues. In an embodiment disclosed herein, the polypeptide is attached to the cell penetrating agent via a linker. In an embodiment disclosed herein, the linker is specifically cleaved by a matrix metalloproteinase (MMP). In an embodiment disclosed herein, the linker is specifically cleaved by MMP2. In an embodiment disclosed herein, the linker comprises the amino acid sequence PLGC(Me)AG (SEQ ID NO: 2). In an embodiment disclosed herein, the linker consists of the amino acid sequence PLGC(Me)AG (SEQ ID NO:2). In an embodiment disclosed herein, the therapeutic moiety comprises a compound capable of inhibiting or activating extracellular matrix turnover. In an embodiment disclosed herein, the therapeutic moiety comprises a compound capable of inhibiting or activating matrix metalloproteinase (MMP) activity. In an embodiment disclosed herein, the compound is capable of inhibiting MMP activity. In an embodiment disclosed herein, the compound is an MMP14 inhibitor. In an embodiment disclosed herein, the MMP14 inhibitor is Naphthofluorescein. In an embodiment disclosed herein, the therapeutic moiety further comprises a carrier. In an embodiment disclosed herein, the carrier is a nanoparticle. In an embodiment disclosed herein, the carrier is a poly(2-diisopropylaminoethyl methacrylate (PDPA) nanoparticle.

In another aspect of the present disclosure, there is provided a method of treating or preventing a condition associated with unwanted extracellular matrix turnover in a subject, the method comprising administering to a subject in need thereof a therapeutic construct as herein described.

In an embodiment disclosed herein, the condition is selected from the group consisting of: cardiovascular disease, atherosclerosis, cardiac remodelling post-myocardial infarction, stroke, cerebrovascular disease, peripheral artery disease, restenosis, diabetic nephropathy, glomerulonephritis, human crescentic glomerulonephritis, IgA nephropathy, membranous nephropathy, lupus nephritis, pancreatitis, vasculitis, rheumatoid arthritis, osteoarthritis, allograft rejection, systemic sclerosis, neurodegenerative disorders and demyelinating disease, multiple sclerosis, Alzheimer's disease, chronic obstructive pulmonary disease, asthma, neuropathic pain, inflammatory pain, and cancer. In an embodiment disclosed herein, the condition is selected from the group consisting of atherosclerosis cardiac remodelling post-myocardial infarction and stroke.

### BRIEF DESCRIPTION OF THE DRAWINGS

Embodiments of the disclosure are described herein, by way of non-limiting example only, with reference to the following drawings:

FIG. 1 shows the results of the mass spectrum analysis of the T-peptide-LPETG peptide.

FIG. 2 shows the results of the mass spectrum analysis of T-peptide-ACPP-alkyne peptide.

FIG. 3 shows the results of the mass spectrum analysis of the GGG-T-peptide-ACPP-cysteine peptide.

FIGS. 4A-C are representative photomicrographs of tissue sections of atherosclerotic plaques of a mouse carotid artery. FIG. 4A shows binding of the T-peptide-FITC conjugate to a sections of an unstable atherosclerotic plaque. FIG. 4B shows the absence of binding of the control peptide-FITC conjugate to the vascular lumen of a tandem section of unstable atherosclerotic plaque. FIG. 4C shows the absence of binding of the T-peptide-FITC conjugate to a stable atherosclerotic plaque within a mouse carotid artery: IEL: Internal elastic lamina, MEL: Middle elastic lamina, EEL: External elastic lamina. Fluorescence microscopy in FITC channel with bright field overlay (20× Objective).

FIGS. 5A-C are representative photomicrographs of tissue sections of atherosclerotic plaques of a mouse carotid artery. FIG. 5A shows an unstable carotid plaque after intravenous administration with T-peptide-FITC. FIG. 5B shows an unstable carotid plaque after intravenous administration with control-peptide-FITC. FIG. 5C shows a stable aortic arch plaque after intravenous administration with T-peptide-FITC: internal elastic lamina (IEL), middle elastic lamina (MEL) and external elastic lamina (EEL); overlay image from fluorescence microscopy in the FITC channel at 20× objective.

FIGS. 6A-D are representative photomicrographs of glass slides coated with undigested collagen IV or MMP2-predigested collagen IV following incubation with either the T-peptide-FITC conjugate (FIGS. 6A, B and D) or the control peptide-FITC conjugate (FIG. 6C). FIG. 6A is an MMP2-predigested collagen IV coated glass slide that was incubated with the T-peptide-FITC conjugate. FIG. 6B is an undigested collagen IV coated glass slide that has been incubated which shows the T-peptide-FITC conjugate. FIG. 6C shows an MMP2-predigested collagen IV coated glass slide that was incubated with the control peptide-FITC conjugate. FIG. 6D shows an MMP2 pre-digested collagen IV coated glass slide that was incubated with unlabelled T-peptide, followed by the T-peptide-FITC conjugate; fluorescence microscopy in FITC channel with bright field overlay (4× objective). Representative photomicrographs from n=3 are shown. Scale bar=300 mm.

FIG. 7A shows a representative photomicrograph of a human carotid plaque showing binding of the T-peptide-GFP conjugate onto parts of the plaque. FIG. 7B shows a representative photomicrograph of a human carotid plaque showing no evidence of binding of the control peptide (GGG)-GFP conjugate to the plaque; Fluorescence microscopy in the FITC channel at 4× objective; FC=Fibrous cap.

FIG. 8 is a schematic of the attachment of the collagen-homing T-peptide to an activatable cell penetrating peptide (ACPP). The ACPP comprises a polycationic sequence of 9 D-arginine zippered to a polyanionic sequence of 8 D-glutamic acid held together by a U-shaped peptide linker (5-amino-3-oxapentanoyl flexible hydrophilic linker) that contains an amino acid sequence of PLGC(Me)AG that is preferentially cleaved by MMP2. A drug carrying polymer nanosponge is attached to the polycationic arm of the ACPP by the thiolene-click reaction and subsequently loaded with Naphthofluorescein.

FIG. 9 shows a nuclear magnetic resonance (NMR) spectrum after the attachment of an aldehyde linker to a drug carrying polymer nanosponge.

FIG. 10 shows a nuclear magnetic resonance (NMR) spectrum after the attachment of the T-peptide to a drug carrying polymer nanosponge.

FIG. 11 shows a nuclear magnetic resonance (NMR) spectrum after the attachment of a Cy3 hydrazide dye to a drug carrying polymer nanosponge.

FIG. 12 shows ACPP cell entry after the addition of MMP2. ACPP tagged with SHIP probe shows intracellular TRITC signal upon cell entry of the polycationic arm (scale bar 10 mm). (A) DIC overlay, (B) fluorescence signal.

FIG. 13 shows the effect of acid-treated Naphthatluorescein on MMP14 inhibition in HT1080 cells.

FIG. 14 shows confocal images of HT1080 cells and uptake of Cy3 (red) labelled nanosponge. Image is taken in the Z stack middle of the cell layer showing intracellular signal. Confocal microscopy of RAW cells to investigate lysosomes location. (b) 2D reconstruction of Z stack confocal images of RAW cells with uptake of Cy3 (red) labelled nanosponge are shown as red dots while the lysosomes are in blue. (c) 3D snap shot showing that the red and blue dots are spatially apart. Scale bar=8 μm.

FIG. 15 shows polarised light microscopy of a carotid plaque section from mouse in vivo study (Picrosirius Red stained) (a) Mouse treated with targeted drug construct shows abundant collagen compared to control mice treated with (b) PBS, (c) free drug and (d) empty nanosponge. Scale bars=100 μm. Confocal images of HT1080 cells and uptake of Cy3 (red) labelled.

FIG. 16 shows collagen content as expressed as percentage of the plaque area showing marked differences between mouse treatment groups. ns=not significant; *=p<0.05.

FIG. 17 shows photomicrographs of fluorescent microscopy showing T-peptide-FITC specifically binding to areas of myocardial fibrosis in the infarcted mouse myocardium induced by experimental LAD (A). No binding of the control peptide-FITC conjugate to areas of myocardial fibrosis was evident (B).

FIG. 18 shows electrospray ionisation (ESI) mass spectrometry scans for (A) the S-peptide-MeCOSar conjugate (control) and (B) the T-peptide-MeCOSar conjugate.

FIG. 19 shows PET/CT imaging with a ⁶⁴Cu-T-peptide-MeCOSar tracer binding to areas of fibrosis in the right atrium (RA) of rapidly paced sheep heart, with no signal detected in the healthy right ventricle (RV).

FIG. 20 shows PET imaging of fibrosis in a mouse heart failure model (Dtg) as compared to control animals (Ntg) using a ⁶⁴Cu-T-peptide-MeCOSar tracer or the scrambled control peptide tracer (s-peptide/Dtg).

### DETAILED DESCRIPTION

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The articles “a” and “an” are used herein to refer to one or to more than one (i.e., to at least one) of the grammatical object of the article. By way of example, “an agent” means one agent or more than one agent.

In the context of this specification, the term “about” is understood to refer to a range of numbers that a person of skill in the art would consider equivalent to the recited value in the context of achieving the same function or result.

### Imaging Agent

As noted elsewhere herein, the present disclosure is predicated on the inventors' surprising finding that the polypeptide TLTYTWS (SEQ ID NO:1) can differentially bind to unstable atherosclerotic plaques and areas of collagen turnover (fibrosis) in vitro, ex vivo and in vivo. By using this polypeptide as part of a targeting moiety, the inventors have developed a novel imaging agent for identifying unstable atherosclerotic plaques and fibrosis in vivo for diagnostic and/or prognostic purposes.

Accordingly, in one aspect of the present disclosure, there is provided an imaging agent comprising a polypeptide linked to a detectable label, wherein the polypeptide comprises the amino acid sequence TLTYTWS (SEQ ID NO:1).

As used herein the term “polypeptide” means a polymer made up of amino acids linked together by peptide bonds. It would be understood by persons skilled in the art that the polypeptide of SEQ ID NO:1 may comprise one or more additional amino acid residues attached to its N-terminus and/or C-terminus without diminishing the ability of the polypeptide to specifically bind to unstable atherosclerotic plaques and areas of fibrosis in vitro or in vivo. Polypeptides modified in this way are also referred to herein as variants. In an embodiment disclosed herein, a variant of the polypeptide of SEQ ID NO: I comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 additional amino acids at its N-terminus and/or C-terminus. It is also well within the ability of persons skilled in the art to ascertain whether or not the addition of one or more additional amino acid residues to the N-terminus and/or C-terminus of the polypeptide of SEQ ID NO:1 will diminish the ability of the modified polypeptide to specifically bind to unstable atherosclerotic plaques and areas of fibrosis in vitro or in vivo. Illustrative examples of suitable methods of determining the ability of a polypeptide variant, as herein described, to specifically bind to unstable atherosclerotic plaques and areas of fibrosis in vitro or in vivo are disclosed elsewhere herein (see Examples section).

A variant may also result from amino acid substitutions that are functionally equivalent; that is, amino acid substitutions that, when effected, result in a polypeptide that retains at least some of the specificity of the parent polypeptide of SEQ ID NO:1, such as its ability to specifically bind to MMP2-digested collagen IV, as described elsewhere herein. Persons skilled in the art can readily determine whether a variant of the polypeptide of SEQ ID NO:1 retains at least some of the function of the parent molecule (i.e., of SEQ ID NO:1) by using any suitable method know in the art, illustrative examples of which include the in vitro, ex vivo and in vivo methods described elsewhere herein (see Examples section). In an embodiment disclosed herein, the variant is derived from conservative amino acid substitution. By “conservative amino acid substitution” is meant the replacement of an amino acid residue with a different amino acid residue of approximately equivalent size, charge and/or polarity as the amino acid residue being replaced. Illustrative example of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

In an embodiment disclosed herein, the polypeptide consists of the amino acid sequence TLTYTWS (SEQ ID NO:1).

The polypeptide comprising the amino acid sequence TLTYTWS (SEQ ID NO:1) is also referred to interchangeably herein as a “targeting peptide” or “targeting moiety”; that is, a compound that, after administration to a subject, is taken up selectively by, or localises at, a particular site of the subject in vivo.

### Detectable Label

Suitable detectable labels will be known to persons skilled in the art, illustrative examples of which include a radio-isotope, an imaging dye, a paramagnetic material or a microbubble. In an embodiment disclosed herein, the detectable label is selected from the group consisting of a radio-isotope, an imaging dye, a paramagnetic material or a microbubble.

It will be understood by persons skilled in the art that the choice of detectable label will depend on the method that will be employed to detect the imaging agent. For example, where the imaging agent is to be used to detect unstable atherosclerotic plaques or areas of fibrosis in vitro or ex vivo, it may be desirable to use an imaging dye such as a fluorophore. Suitable fluorophores will be known to persons skilled in the art, illustrative examples of which include fluorescein (FITC). Cy3, Cy3.5, Cy5 and Cy3.5. Where the imaging agent is to be used to detect unstable atherosclerotic plaques or areas of fibrosis in vivo, it may be desirable to use a contrasting agent such as a radiolabel or radio-isotope. Thus, in an embodiment disclosed herein, the detectable label is a radio-isotope.

The terms “radiolabel”, “radio-isotope” and the like are used herein to denote a compound where one or more atoms are replaced or substituted by an atom having an atomic mass or mass number different from the atomic mass or mass number typically found in nature (i.e., naturally occurring). In an embodiment, the detectable label is a radio-isotope selected from the group consisting of carbon-11, nitrogen-13, oxygen-14, oxygen-15, fluorine-18, iron-52, copper-62, copper-64, zinc-62, zinc-63, gallium-68, arsenic-74, bromine-76, rubidium-82, yttrium-86, zirconium-89, technetium-94m, indium-110m, iodine-122, iodine-123, iodine-124, iodine-131, or cesium-137. In some configurations, a radionuclide can be selected from carbon-11, nitrogen-13, oxygen-15, fluorine-18, iron-52, copper-64, gallium-68, yttrium-86, bromine-76, zirconium-89, iodine-123, and iodine-124.

Copper-64 (⁶⁴Cu) has a long half-life (about 12.7 hours) and positron energy that results in high quality images and is therefore a promising alternative isotope for PET imaging. The longer half-life of ⁶⁴Cu has advantages relating to centralised production and distribution of PET tracers, as well as allowing imaging at longer time points, often resulting in better signal to noise ratios. Thus, in an embodiment disclosed herein, the radio-isotope is ⁶⁴Cu.

In some embodiments, more than one detectable label can be used.

The detectable label can be linked to the polypeptide by any suitable method of conjugation. Suitable methods of conjugating the detectable label to the polypeptide disclosed herein will be known to persons skilled in the art. Illustrative examples of suitable methods of conjugating the detectable label to the polypeptide are described elsewhere herein (see Examples section). The choice of conjugation method may depend on the detectable label to be employed.

In some embodiments, methods of conjugating the detectable label to the polypeptide disclosed herein will require a linker to be attached to the N-terminus or C-terminus of the polypeptide of SEQ ID NO:1 or variant thereof to facilitate conjugation. Suitable linkers will be known to persons skilled in the art, illustrative examples of which include polypeptides comprising N-terminal LPETG, N-terminal ACPP-alkyne and C-terminal GGG, or any combination thereof.

### Complexing Agent

In some embodiments, it may be desirable to attach the detectable label to a complexing agent to maximise retention of the detectable label to the imaging agent and thereby minimise loss or degradation of the detectable label from the imaging agent, in particular under physiological conditions. Thus, in an embodiment disclosed herein, the detectable label is attached to a complexing agent for the retention of the detectable label within the imaging agent. Suitable complexing agents for this purpose will be known to persons skilled in the art, an illustrative example of which is 1,4,7,10-tetraazacyclododecane-1,4,7,10-tetraacetic acid (DOTA). Thus, in an embodiment disclosed herein, the complexing agent is 1,4,7,10-tetraazacyclododecane-1,4,7,10-tetraacetic acid (DOTA).

The present inventors have surprisingly found that chelating agents based on a hexaaminemacrobicyclic cage structure, such as 5-(8-methyl-3,6,10,13,16,19-hexaaza-bicyclo[6.6.6]icosan-1-ylamino)-5-oxopentanoic acid (MeCOSar), bind radiolabels such as ⁶⁴Cu with fast complexation kinetics at room temperature to form complexes with high thermodynamic and kinetic stability that are stable under physiological conditions. Thus, in an embodiment disclosed herein, the complexing agent is 5-(8-methyl-3,6,10,13,16,19-hexaaza-bicyclo[6.6.6]icosan-1-ylamino)-5-oxopentanoic acid (MeCOSar).

MeCOSar is a bifunctional chelator that forms copper complexes of exceptional stability by virtue of a cage amine (sarcophagine) ligand. In contrast to first generation ligands such as (DOTA), MeCOSar does not require specific metal-free production conditions beyond the GMP-certified generation and purification of the targeting peptide and its coupling to the MeCOSar (see Paterson el al, Dalton Trans. 2014, 21;43(3):1386-96). The MeCOSar-based imaging agent developed by the present inventors was surprisingly found to have the unique property of selectively binding to unstable atherosclerotic plaques and areas of fibrosis in viva, while retaining high thermodynamic and kinetic stability under physiological conditions.

### Method of Detection

As noted elsewhere herein, the present disclosure is predicated on the inventors' surprising finding that the polypeptide TLTYTWS (SEQ ID NO:1) can differentially bind to unstable atherosclerotic plaques and areas of collagen turnover (fibrosis) in vitro, ex vivo and in vivo. Thus, also enabled herein are methods of detecting unstable atherosclerotic plaques and areas of collagen turnover (fibrosis) in vitro, ex vivo or in vivo for the diagnosis and/or prognosis of conditions associated with unstable atherosclerotic plaques and fibrosis.

In an aspect of the present disclosure, there is provided a method of detecting an atherosclerotic plaque in a vascular lumen of a subject in vivo, the method comprising: a) administering to a subject in need thereof the imaging agent as herein described; and b) detecting the detectable label of the imaging agent, wherein the presence of the detectable label within the vascular lumen of the subject is indicative of binding of the imaging agent to an unstable atherosclerotic plaque.

In another aspect of the present disclosure, there is provided a method of detecting fibrosis in a subject in vivo, the method comprising: a) administering to a subject in need thereof the imaging agent as herein described; and b) detecting the detectable label of the imaging agent, wherein the presence of the detectable label in the subject is indicative of binding of the imaging agent to an area of fibrosis.

Suitable methods of detecting the detectable label in a subject in vivo, also referred to herein as “in vivo imaging”, will be familiar to persons skilled in the art and typically refer to non-invasive methods of generating an image or a series of images of all or part of a subject, more particularly an internal aspect of the subject. It will be understood that the choice of method will depend on the type of detectable label(s) being used. Suitable imaging techniques will be known to persons skilled in the art, illustrative examples of which include single photon emission computed tomography, positron emission tomography (PET); near infrared fluorescence imaging, ultrasound imaging and magnetic resonance imaging. In an embodiment disclosed herein, the method for detecting the detectable label is selected from the group consisting of: single photon emission computed tomography; positron emission tomography; near infrared fluorescence imaging; ultrasound imaging; and magnetic resonance imaging.

The strength of nuclear imaging is its quantitative information at a functional level with a signal sensitivity that is significantly higher than that of MRI or CT. Currently, the most promising modality in nuclear medicine is PET, which acquires images in humans with a spatial resolution in the mm range, thereby improving on the performance of single photon emission computed tomography (SPECT), which has a resolution of 10-15 mm. PET is significantly more sensitive than SPECT, as a collimator blocks 99% of all incoming radiation within the SPECT detector ring. In addition, PET is inherently quantitative and gives a precise activity distribution within tissue something not possible in SPECT. Thus, in an embodiment disclosed herein, the method for detecting the detectable label is positron emission tomography (PET).

As described elsewhere herein, the presence of the detectable label within the vascular lumen of the subject to whom the imaging agent is administered is indicative of the binding of the imaging agent to an unstable atherosclerotic plaque. As is also described elsewhere herein, the presence of the detectable label in the subject to whom the imaging agent is administered is indicative of the binding of the imaging agent to an area of fibrosis, also referred to herein as a scar or a fibrotic lesion. In some embodiments, it may be desirable to perform in vivo imaging immediately or at least shortly after the imaging agent is administered to the subject, as the strength of the signal provided by the detectable label may diminish overtime; for example, by clearance of the imaging agent from the subject over time and/or by the gradual loss of the detectable label from the imaging agent under physiological conditions following administration of the imaging agent to the subject. In some embodiments, in vivo imaging is performed within at least 30 minutes, within at least 1 hour, within at least 2 hours, within at least 3 hour, within at least 4 hours, within at least 6 hours, within at least 7 hours, within at least 8 hours, within at least 9 hours or within at least 10 hours following administration of the imaging agent to the subject. The optimal period within which to perform in vivo imaging following administration of the imaging agent to the subject will be known or can at least be determined by persons skilled in the art without undue experimentation, and will generally depend on the type of detectable label(s) being employed.

It will be understood by persons skilled in the art that the presence of a higher signal generated by the detectable label within the vascular lumen of the subject to whom the imaging agent is administered is indicative of the binding of the imaging agent to an unstable atherosclerotic plaque. This is in constrast to any background level of detectable label that may be detected by in vivo imaging. Similarly, it will be understood by persons skilled in the art that the presence of a higher signal generated by the detectable label within the subject to whom the imaging agent is administered is indicative of the binding of the imaging agent to an area of fibrosis, in constrast to any background level of detectable label that may be detected by in vivo imaging.

It will be understood that the imaging agent disclosed herein is also applicable to a method of detecting unstable atherosclerotic plaques and fibrosis in a subject ex vivo. For example, a tissue sample suspected of comprising an atherosclerotic plaque or a fibrotic lesion may be obtained from the patient (e.g., via biopsy) and analysed for the presence or absence of an unstable atherosclerotic plaque and/or a fibrotic lesion in accordance with the methods disclosed herein. Illustrative examples of methods suitable for the ex vivo detection of the detectable label of the imaging agent are given in the Examples section herein. Other suitable methods for the detection of the detectable label of the imaging agent ex vivo will be familiar to persons skilled in the art.

Thus, in another aspect of the present disclosure, there is provided a method of detecting an unstable atherosclerotic plaque or fibrosis in a subject ex vivo, the method comprising: a) contacting a biological tissue sample obtained from a subject with an imaging agent as herein described; and h) detecting the detectable label of the imaging agent, wherein the presence of the detectable label is indicative of binding of the imaging agent to an unstable atherosclerotic plaques or an area of fibrosis in the tissue sample.

The term “subject” as used herein refers to mammals and includes humans, primates, livestock animals (e.g., sheep, pigs, cattle, horses, donkeys), laboratory test animals (e.g., mice, rabbits, rats, guinea pigs), companion animals (e.g., dogs, cats) and captive wild animals (e.g., foxes, kangaroos, deer). In an embodiment disclosed herein, the subject is a human or a laboratory test animal. In an embodiment, the subject is a human, In an embodiment disclosed herein, the subject has or is suspected of having a condition associated with unwanted extracellular matrix turnover (e.g., fibrosis). Condition associated with unwanted extracellular matrix turnover will be known to persons skilled in the art and may include conditions associated with unwanted extracellular matrix deposition or reduced extracellular matrix degradation (e.g., tissue fibrosis) or unwanted extracellular matrix degradation or reduced extracellular matrix deposition (e.g., as seen in unstable atherosclerotic plaques). Illustrative examples of conditions associated with unwanted extracellular matrix turnover include cardiovascular disease, atherosclerosis, cardiac remodelling post-myocardial infarction, stroke, cerebrovascular disease, peripheral artery disease, restenosis, diabetic nephropathy, glomerulonephritis, human crescentic glomerulonephritis, IgA nephropathy, membranous nephropathy, lupus nephritis, pancreatitis, vasculitis, rheumatoid arthritis, osteoarthritis, allograft rejection, systemic sclerosis, neurodegenerative disorders and demyelinating disease, multiple sclerosis, Alzheimer's disease, chronic obstructive pulmonary disease, asthma, endometriosis, neuropathic pain, inflammatory pain, and cancer. Thus, in an embodiment disclosed herein, the condition is selected from the group consisting of: cardiovascular disease, atherosclerosis, cardiac remodelling post-myocardial infarction, stroke, cerebrovascular disease, peripheral artery disease, restenosis, diabetic nephropathy, glomerulonephritis, human crescentic glomerulonephritis, IgA nephropathy, membranous nephropathy, lupus nephritis, pancreatitis, vasculitis, rheumatoid arthritis, osteoarthritis, allograft rejection, systemic sclerosis, neurodegenerative disorders and demyelinating disease, multiple sclerosis, Alzheimer's disease, chronic obstructive pulmonary disease, asthma, endometriosis, neuropathic pain, inflammatory pain, and cancer. In an embodiment disclosed herein, the condition is selected from the group consisting of atherosclerosis, cardiac remodelling post-myocardial infarction, endometriosis and stroke.

### Therapeutic Construct

As noted elsewhere herein, the present inventors have surprising found that the polypeptide TLTYTWS (SLQ ID NO:1) can differentially bind to unstable atherosclerotic plaques and areas of collagen turnover (fibrosis) in vitro, ex vivo and in vivo. Thus, also enabled herein are therapeutic constructs that utilise the polypeptide of SEQ ID NO:1 or variants thereof, as herein described, as a targeting moiety to deliver a therapeutic agent to unstable atherosclerotic plaques and areas of collagen turnover (fibrosis) in a subject in need thereof. Thus, in another aspect of the present disclosure, there is provided a therapeutic construct comprising a therapeutic moiety linked to a targeting moiety, wherein the targeting moiety comprises a polypeptide comprising the amino acid sequence TLTYTWS (SEQ ID NO: 1).

In an embodiment disclosed herein, the targeting moiety comprises a variant of the polypeptide of SEQ ID NO:1, as described elsewhere herein. In another embodiment disclosed herein, the polypeptide consists of the amino acid sequence TLTYTWS (SEQ ID NO:1).

In an embodiment disclosed herein, the targeting moiety further comprises a cell penetrating agent, the purpose of which is to facilitate passage or entry of the therapeutic moiety into a cell. Suitable cell penetrating agents will be familiar to persons skilled in the art, illustrative examples of which include branched or linear peptides comprising a plurality of positively-charged amino acid residues. Thus, in an embodiment disclosed herein, the cell penetrating agent comprises a plurality of positively-charged amino acid residues. In an embodiment, the cell penetrating agent comprises a plurality of D-arginine residues.

In an embodiment disclosed herein, the polypeptide is attached to the cell penetrating agent via a linker.

In an embodiment, the cell penetrating agent is an activatable cell penetrating agent. By “activatable” is meant that the cell penetrating agent is inactive or inert when administered to the subject, but is activated under specific physiological conditions. For example, the activatable cell penetrating agent may be attached to a moiety (e.g., a linker) that renders the cell penetrating agent inactive, and wherein the moiety is cleaved under physiological conditions to activate the cell penetrating agent and facilitate entry of the therapeutic agent into the cell. In some embodiments, the moiety to which the activatable cell penetrating agent is attached is the linker to which the polypeptide or variant thereof is attached to the cell penetrating agent.

Suitable activatable linkers will be familiar to persons skilled in the art, illustrative examples of which include linkers that are specifically cleaved by matrix metalloproteinases (MMPs). Thus, in an embodiment disclosed herein, the linker is specifically cleaved by a matrix metalloproteinase (MMP). In an embodiment disclosed herein, the linker is specifically cleaved by MMP2. In an embodiment disclosed herein, the linker comprises the amino acid sequence PLGC(Me)AG (SEQ ID NO:2). In an embodiment disclosed herein, the linker consists of the amino acid sequence PLGC(Me)AG (SEQ ID NO:2).

As used herein, the term “therapeutic moiety” includes a compound or chemical structure that is capable of eliciting a therapeutic or prophylactic effect when administered to a subject in need thereof. In an embodiment disclosed herein, the therapeutic moiety comprises a compound capable of inhibiting or activating extracellular matrix turnover. Suitable therapeutic compounds will be familiar to persons skilled in the art, the choice of which will depend on the condition to be treated. For example, where the therapeutic construct is to be used for the treatment of unstable atherosclerotic plaques, it may be desirable to use a therapeutic compound that promotes extracellular matrix deposition, for example, by enhancing the production of extracellular matrix (e.g., collagen) or by reducing extracellular matrix turnover by inhibiting extracellular matrix degradation by MMPs. By promoting extracellular matrix deposition, the otherwise vulnerable atherosclerotic plaques are stabilised, minimising the risk of rupture and the adverse consequences that arise therefrom. Suitable therapeutic compounds that promote extracellular matrix deposition will be familiar to persons skilled in the art, illustrative examples of which include compounds capable of inhibiting MMP activity. Thus, in an embodiment disclosed herein, the therapeutic moiety comprises a compound capable of inhibiting MMP activity. Such compounds may inhibit MMP activity by inhibiting or otherwise reducing the expression or release of MMPs from a cell or population of cells. Suitable MMP inhibitors will be familiar to persons skilled in the art, illustrative examples of which include Nelfinavir, Tanomastat (4-(4′-Chlorobiphenyl-4-yl)-4-oxo-2-[(phenylsulfanyl)methyl]butanoic acid), Saquinavir, Indinavir, BB-1101, Doxycycline, Marimastat (N-[12,2-dimethyl-1-(methylcarbamoyl)propyl]-2-[hydroxy-(hydroxycarbamoyl) methyl]-4-methyl-pentanamide), TNF-α Protease Inhibitor-0 (TAPI-0; CAS No. 143457-40-3) and structural analogues thereof (e.g., TAPI-1, TAPI-2), Phosphoramidon, Luteolin, Alendronate, o-Phenanthroline, 4-epi-Chlortetracycline, Actinonin, DL-Thiorphan, 4-epi-Demeclocycline, Zinc methacrylate, Funalenone, Keracyanin and Naphthofluorescein

In an embodiment disclosed herein, the compound is an MMP14 inhibitor. Suitable MMP14 inhibitors will be familiar to persons skilled in the art, an illustrative example of which is Naphthofluorescein. In an embodiment disclosed herein, the MMP14 inhibitor is Naphthofluorescein.

For sonic conditions, it may be desirable to use a therapeutic compound that inhibits extracellular matrix deposition, for example, by inhibiting the production of extracellular matrix (e.g., collagen) or by enhancing extracellular matrix turnover by promoting extracellular matrix degradation by MMPs. This approach may be particularly suited to conditions associated with tissue fibrosis, such as the fibrosis that develops in myocardial tissue post-myocardial infarction (MI) or during chronic heart failure independent of MI. By inhibiting extracellular matrix deposition, fibrosis within the tissue is minimised, the result of which is an improvement of tissue integrity and function. Suitable therapeutic compounds that inhibit extracellular matrix deposition will be familiar to persons skilled in the art, illustrative examples of which include Pirfenidone, Relaxin, Galectin-3, modified citrus pectin, N-acetylcysteine, corticosteroids, Azathioprine, Methotrexate, Cyclophosphamide, the anti-TGF beta 1 monoclonal antibody CAT-192 (Metelimumab), Decorin, Imatinib ((4-methylpiperazin-1-yl)methyl]-N-(4-methyl-3-{[4-(pyridin-3-yl) pyrimidin-2-yl]amino}phenyl)benzamide), Dasatinib (BMS-354825), Nilotinib (4-methyl-N-[3-(4-methyl-1H-imidazol-1-1-yl)-5-(trifluoromethyl)phenyl]-3-[(4-pyridin-3-ylpyrimidin-2-yl)amino]benzamide) tranilast, tranilast derivatives (e.g., FT011; Fibrotech Therapeutics) and the anti-fibrotic compounds described in U.S. Pat. Nos. 8,765,812, 8,652,540 and 8,283,323. In an embodiment disclosed herein, the therapeutic moiety comprises a radioisotope, illustrative examples of which are described elsewhere herein.

In an embodiment disclosed herein, the therapeutic moiety further comprises a carrier. Suitable carriers will be familiar to persons skilled in the art, illustrative examples of which include polymeric nanoparticles, dendrimers, carbon nanotubes, gold nanoparticles, liposomes and micelles. In an embodiment disclosed herein, the carrier is a nanoparticle. Suitable nanoparticles will be familiar to persons skilled in the art, illustrative examples of which include poly(2-diisopropylaminoethyl methacrylate (PDPA) nanoparticles. Nanoparticles are currently being developed for biomedical applications such as sensing, bio-reactions and drug delivery. In order for these applications to be carried out, nanoparticles generally need to be loaded with functional cargo, such as liposomes, enzymes, or therapeutic compounds and targeted to the specific site of action, such as organs. It is desirable that the functional cargo be preferentially retained in the nanoparticle either by using impermeable material or by conjugating the cargo to the nanoparticle shell. Conjugation offers some advantages, such as chemical control over the linking moieties, which can be pH, redox or enzyme responsive, while impermeable nanoparticles offer other advantages such as higher drug loading and responsiveness based on their shell properties, such as enzymatic degradation, and pH- or salt-induced swelling. Besides the retention of functional cargo, nanoparticles generally need a controlled surface for biomedical applications, such as stealth and targeting functionalities for site-specific drug delivery. By controlling the surface of the nanoparticle, the interaction between the particle and its surrounding environment is also controlled, and therefore specific functions that arise from the encapsulated or conjugated cargo can be localized to specific environments that are targeted by the nanoparticle. Therefore, cargo retention and proper localization are two further considerations for effective in vivo drug delivery. The present inventors have developed a polymer platform with high drug loading and small-molecule retention that for the in vivo targeted delivery of therapeutic-loaded particles to areas of extracellular matrix turnover, such as unstable atherosclerotic plaques.

In an embodiment disclosed herein, the carrier is a poly(2-diisopropylaminoethyl methacrylate (PDPA) nanoparticle. In some embodiments, it may be desirable to functionalise the nanoparticle to enhance its stability, particularly under physiological conditions.

### Methods of Treatment

Current options for treating unstable atherosclerotic plaques or fibrosis are either highly invasive (catheter balloon and stenting) or require life-long, high-dose medication resulting in considerable side effects. Ideally, delivery of a therapeutic compound to unstable atherosclerotic plaques or areas of tissue fibrosis must be specific to avoid off target effects to healthy tissue. In addition, as extracellular matrix deposition and turnover are ongoing processes in the development of unstable atherosclerotic plaques and fibrotic lesions, a sustained level of therapeutic compound over a long period of time is generally required. Current therapeutic options such as systemic statin treatment shows promising results in secondary prevention, but fail to treat highly inflamed plaques responsible for acute thrombotic events.

The methods disclosed herein solve or at least partly alleviate such problems by providing an improved way of treating unstable atherosclerotic plaques and fibrotic lesions by allowing for the targeted delivery of a therapeutic agent specifically to unstable plaques and areas of fibrosis, thus avoiding the adverse off-target effects to healthy tissue that are often associated with existing treatments.

Thus, in another aspect of the present disclosure, there is provided a method of treating a condition associated with unwanted extracellular matrix turnover in a subject, the method comprising administering to a subject in need thereof the therapeutic construct as herein described.

As used herein, the term “extracellular matrix turnover” includes extracellular matrix deposition and degradation. Extracellular matrix material includes, but is not limited to, collagen (e.g., collagen I, II, III and IV) and fibronectin.

Conditions associated with unwanted extracellular matrix turnover will be known to persons skilled in the art and may include conditions associated with unwanted extracellular matrix deposition or reduced extracellular matrix degradation (e.g., tissue fibrosis) or unwanted extracellular matrix degradation or reduced extracellular matrix deposition (e.g., as seen in unstable atherosclerotic plaques). Illustrative examples of conditions associated with unwanted extracellular matrix turnover include cardiovascular disease, atherosclerosis, cardiac remodelling post-myocardial infarction, stroke, cerebrovascular disease, peripheral artery disease, restenosis, diabetic nephropathy, glomerulonephritis, human crescentic glomerulonephritis, IgA nephropathy, membranous nephropathy, lupus nephritis, pancreatitis, vasculitis, rheumatoid arthritis, osteoarthritis, allograft rejection, systemic sclerosis, neurodegenerative disorders and demyelinating disease, multiple sclerosis, Alzheimer's disease, chronic obstructive pulmonary disease, asthma, endometriosis, neuropathic pain, inflammatory pain, and cancer.

Thus, in an embodiment disclosed herein, the condition is selected from the group consisting of: cardiovascular disease, atherosclerosis, cardiac remodelling post-myocardial infarction, stroke, cerebrovascular disease, peripheral artery disease, restenosis, diabetic nephropathy, glomerulonephritis, human crescentic glomerulonephritis, IgA nephropathy, membranous nephropathy, lupus nephritis, pancreatitis, vasculitis, rheumatoid arthritis, osteoarthritis, allograft rejection, systemic sclerosis, neurodegenerative disorders and demyelinating disease, multiple sclerosis, Alzheimer's disease, chronic obstructive pulmonary disease, asthma, endometriosis, neuropathic pain, inflammatory pain, and cancer.

In an embodiment disclosed herein, the condition is selected from the group consisting of atherosclerosis, cardiac remodelling post-myocardial infarction, endometriosis and stroke.

In the context of this specification, the term “activity” as it pertains to a protein means any cellular function, action, effect or influence exerted by the protein, either by a the protein or any fragment thereof. The cellular function, action, effect or influence may be effected by the protein may be exerted directly or indirectly.

It will be generally understood that the therapeutic methods described herein comprise the step of administering to the subject in need thereof a therapeutically effective amount of the therapeutic construct. As used herein the term “therapeutically effective amount” includes within its meaning a non-toxic but sufficient amount or dose of the construct to provide the desired effect. The exact amount or dose required will vary from subject to subject depending on factors such as the species being treated, the age and general condition of the subject, the severity of the condition being treated, the therapeutic compound or agent being administered, the mode of administration and so forth. Thus, it is not possible to specify an exact “therapeutically effective amount”. However, for any given case, an appropriate “therapeutically effective amount” may be determined by one of ordinary skill in the art using only routine experimentation.

As used herein the terms “treat”, “treating” and “treatment”, refer to any and all uses which remedy a condition or symptom, prevent the establishment of a condition or disease, or otherwise prevent, hinder, retard, or reverse the progression of a condition or disease or other undesirable symptoms in any way whatsoever. Thus, the terms “treat”, “treating” and “treatment” are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms. In the context of some disorders, methods of the present disclosure involve “treating” the disorder in terms of reducing or ameliorating the occurrence of a highly undesirable event associated with the disorder or an irreversible outcome of the progression of the disorder but may not of itself prevent the initial occurrence of the event or outcome. Accordingly, treatment includes amelioration of the symptoms of a particular disorder or preventing or otherwise reducing the risk of developing a particular disorder.

Therapeutic constructs described herein may be administered in accordance with the present disclosure in the form of pharmaceutical compositions, whith typically comprise one or more pharmaceutically acceptable carriers, excipients or diluents. Such compositions may be administered in any convenient or suitable route such as by parenteral routes (e.g., intravenous). In an embodiment, administration is via an intravenous route.

Single or multiple administrations can be carried out with dose levels and pattern being selected by the treating physician. A broad range of doses may be applicable. In some embodiments, an effective amount for a human subject lies in the range of about 4 to about 6 mg/kg body weight. It is to be understood, however, that dosage regimens may be adjusted to provide the optimum therapeutic response. For example, several divided doses may be administered daily, weekly, monthly or other suitable time intervals or the dose may be proportionally reduced as indicated by the exigencies of the situation.

Compositions may be conveniently presented in unit dosage form and prepared by any of the methods well known in the art of pharmacy. The method may include the step of bringing the components of the composition into association with a carrier, e.g. a liquid carrier or finely divided solid carrier, which constitutes one or more accessory ingredients.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl poi ysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethyl-cellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Pharmaceutical forms suitable for injectable use include sterile aqueous solutions (where water soluble) or dispersions and sterile powders for the extemporaneous preparation of sterile injectable solutions or dispersions. The formulation must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the therapeutic construct in the required amount in the appropriate solvent with various other ingredients enumerated above, as required, followed by filter sterilisation.

The present disclosure also contemplates combination therapies, wherein agents the subject of the present disclosure is co-administered with other suitable agents which may facilitate the desired therapeutic or prophylactic outcome. By “co-administered” is meant simultaneous administration in the same formulation or in two different formulations via the same or different routes or sequential administration by the same or different routes. By “sequential” administration is meant a time difference of from seconds, minutes, hours or days between the administration of the two types of agent. Administration may be in any order.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure is further described by reference to the following non-limiting examples.

### EXAMPLES

### Example 1: Synthesis of the T-Peptide

Several versions of the T-peptide (TLTYTWS; SEQ ID NO:1) were synthesized by GL Biochem (Shanghai) Ltd using solid phase peptide synthesis, as described below. Peptide sequences were prepared for T-peptide-LPETG (SEQ ID NO:15), T-peptide-ACPP-alkyne (SEQ ID NO:3) and GGG-T-peptide-ACPP-cysteine (SEQ ID NO:4). These versions allowed for testing the site specific bioconjugation methods using the sortase method (which requires the LPETG sequence; SEQ ID NO:5), copper click reaction (using the alkyne group) and thiol click (using the cysteine functional SH group) and assess whether additional amino acids at the N terminus of the T-peptide sequence will cause steric hindrance of target recognition and/or whether the amino group of the leading threonine residue is important in binding of the T-peptide to its target. The third peptide version (GGG-T-peptide-ACPP-cysteine) was prepared to assess whether additional amino acids at the N terminus of the T-peptide sequence will cause steric hindrance of target recognition, while the cysteine at the C-terminus allowed for thiol click bioconjugation. The addition of amino acid residues at the N terminus of the T-peptide sequence did not reduce or eliminate binding of the T-peptide to MMP2-predigested collagen IV fibres.

1. Method of Synthesis of T-Peptide-LPETG:

Peptide Sequence:

(SEQ ID NO: 6)
HTLTYTWSGGGLPETGGHHHHHH-OH

Materials Used

Amino acids:

### Fmoc-His(trt)-OH

### Fmoc-Pro-OH

### Fmoc-Glv-OH

### Fmoc-Leu-OH

### Fmoc-Ser(tbu)-OH

### Fmoc-Trp(Boc)-OH

### Fmoc-Thr(tbu)-OH

### Fmoc-Tyr(tbu)-OH

### Resin: H-Cys-2Cl -Trt-Cl -Resin

Coupling reagent and base: 2-(1H-Benzotriazole-1-yl)-1,1,3,3-Tetramethyluronium hexafluorophosphate (HBTU), N-methylmorpholine (NMM)
Solvents: DMF, DCM, methanol, piperidineo

Other Reagents used for the Synthesis

Kaiser test solution:
A: 80% phenol+20% anhydrous ethanol
B: distilled pyridine
C: 5g ninhydrin+100 mlL anhydrous ethanol
Fmoc deprotection solution:
20% piperidine in DMF
The cleavage solution:
87.5% TFA+5% phenyl methyl thioanisole+2.5% phenol+2.5% EDT+2.5% H2O

Procedure for the Synthesis of the Peptide

a. Preparing the resin: The weighed H-Cys-2Cl-Trt-Cl-Resin is allowed to swell in DCM for 30 min and then dried under vacuum.
b. Preparing amino acid and HBTU solution for coupling reaction: dissolve 3 eq of amino acid and 2.85 eq of HBTU in DMF.
c. Coupling reaction: add the solution of the amino acid and HBTU in DMF into the resin and then add 6 eq of N-methylmorpholine (NMM). The resin is agitated using a stream of N2 for 30 min.
d. Washing: remove the reaction solution by suction and wash the resin by DMF (3×2 min).
e. Kaiser test: place small amount of resin after the coupling reaction in a test tube, add the Kaiser test solution (of A, B and C) 2 drops each. Heat the resin/solution at 110° C. for 3 min. If the solution is pale and the resin is clear, the coupling reaction is complete.
f. Fmoc Deprotection: The resin is treated with the deproection solution whilst agitated with N2 stream for 30 min, then washed with DMF (6×2 min), dried under vacuum.
g. The steps b to f are repeated till the last amino acid is attached to the peptide.
h. Final wash of the resin: the resin before cleavage requires extensive washing to remove any reagents from the reactions. The resin is washed with methanol (1×2 min), and then DCM (3×2 min), methanol (2×2 min , and then dried under vacuum for 12 h prior to cleavage.

Cleavage of the Peptide to Produce the Crude Peptide

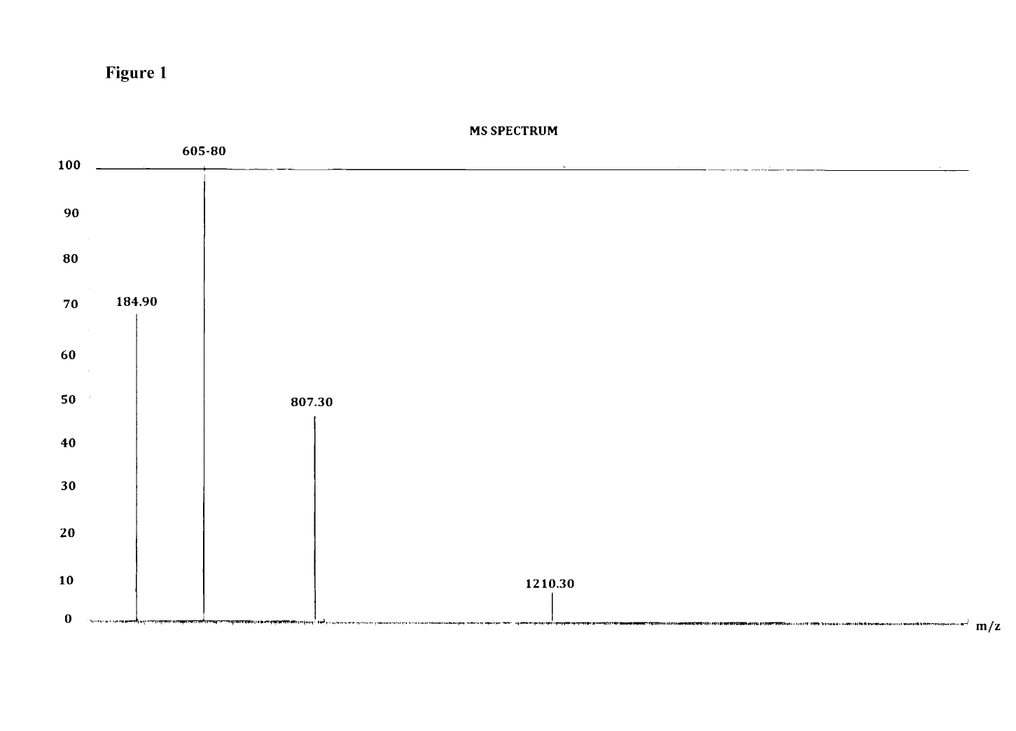

For every 1 g of resin, it requires 10 ml of the cleavage solution. A mixture of the resin and cleavage solution are placed on a shaker at 25° C. for 2 h. The resultant cleavage solution is filtered to remove the resin. To the filtrate (solution) is added 6-8 volume of diethyl ether whilst stirring to principate the crude peptide. The ether mixture is centrifuged and the supernatant is decanted. The peptide is washed with diethyl ether and then the mixture is centrifuged again. The supernatant is discarded. Repeat this washing procedure for 5 times in total to give the crude peptide as white powder. The above washed peptide is dried under vacuum for 24 h. The crude peptide is weighed and then purified by high-performance liquid chromatography (HPLC). The mass spectrum analysis of the T-peptide-LPETG peptide is shown in FIG. 1.

2. Method of Synthesis of T-Peptide-ACPP-Alkyne:

Peptide Sequence:

(SEQ ID NO: 7)
TLTYTWSGLASPAAPAP-eeeeeeee-(Aop)-PLGC(Me)AG-
rrrrrrrrr-GAASPApG

Lower case indicates D-amino acids
Aop=5-amino- 3-oxapentanoyl

### C(Me)=(S-methyl)cysteine

pG=L-Propargylglycine

Materials used

Amino acids:

### Fmoc-Ala-OH

### Fmoc-Pro-OH

### Fmoc-Gly-OH

### Fmoc-Cys (Me)-OH

### Fmoc-Leu-OH

### Fmoc-Aop-OH

### Fmoc-Ser(tbu)-OH

### Fmoc-Trp(Boc)-OH

### Fmoc-Thr(tbu)-OH

### Fmoc-Tyr(tbu)-OH

### Fmoc-D-Arg(pbf)-OH

### Fmoc-D-Glu(Otbu)-OH

### Resin: H-Cys-2Cl-Trt-Cl-Resin

Coupling reagent and base: 2-(1H-Benzotriazole-1-yl)-1,1,3,3-Tetramethyluronium hexafluorophosphate (HBTU), N-methylmorpholine (NMM)
Solvents: DMF, DCM, methanol, piperidine

Other Reagents used for the Synthesis

Kaiser test solution:
A: 80% phenol+20% anhydrous ethanol
B: distilled pyridine
C: 5g ninhydrin+100 mlL anhydrous ethanol
Fmoc deprotection solution:
20% piperidine in DMF
The cleavage solution:
87.5% TFA+5% phenyl methyl thioanisole+2.5% phenol+2.5% EDT+2.5% H2O

Procedure for the Synthesis of the Peptide:

a. Preparing the resin: The weighed H-Cys-2Cl-Trt-Cl-Resin is allowed to swell in DCM for 30 min and then dried under vacuum.
b. Preparing amino acid and HBTU solution for coupling reaction: dissolve 3 eq of amino acid and 2.85 eq of HBTU in DMF.
c. Coupling reaction: add the solution of the amino acid and HBTU in DMF into the resin and then add 6 eq of N-methylmorpholine (NMM). The resin is agitated using a stream of N2 for 30 min,
d. Washing: remove the reaction solution by suction and wash the resin by DMF (3×2 min).
e. Kaiser test: place small amount of resin after the coupling reaction in a test tube, add the Kaiser test solution (of A, B and C) 2 drops each. Heat the resin/solution at 110° C. for 3 min. If the solution is pale and the resin is clear, the coupling reaction is complete.
f. Fmoc Deprotection: The resin is treated with the deprotection solution whilst agitated with N2 stream for 30 min, then washed with DMF (6×2 min), dried under vacuum.
g. The steps b to f are repeated till the last amino acid is attached to the peptide.
h. Final wash of the resin: the resin before cleavage requires extensive washing to remove any reagents from the reactions. The resin is washed with methanol (1×2 min), and then DCM (3×2 min), methanol (2×2 min), and then dried under vacuum for 12 h prior to cleavage.

Cleavage of the Peptide to Produce the Crude Peptide

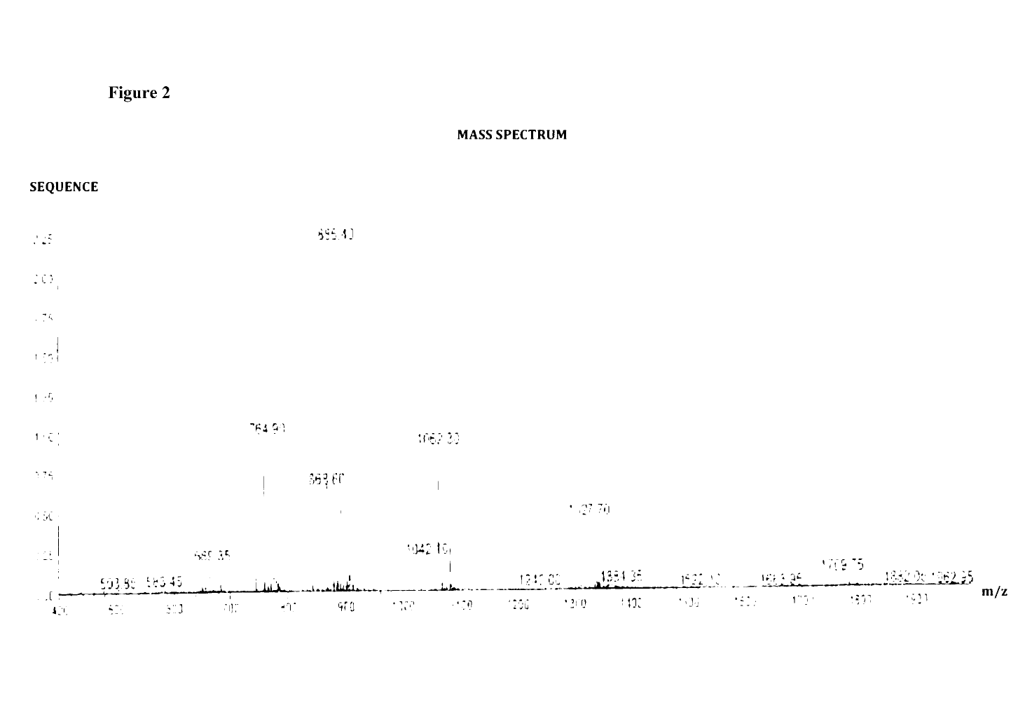

For every 1 g of resin, it requires 10 ml of the cleavage solution. A mixture of the resin and cleavage solution are placed on a shaker at 25° C. for 2 h. The resultant cleavage solution is filtered to remove the resin. To the filtrate (solution) is added 6-8 volume of diethyl ether whilst stirring to principate the crude peptide. The ether mixture is centrifuged and the supernatant is decanted. The peptide is washed with diethyl ether and then the mixture is centrifuged again. The supernatant is discarded. Repeat this washing procedure for 5 times in total to give the crude peptide as white powder. The above washed peptide is dried under vacuum for 24 h. The crude peptide is weighed and then purified by highperformance liquid chromatography (HPLC). The mass spectrum analysis of the T-peptide-ACPP-alkyne peptide is shown in FIG. 2.

3. Synthesis of GGG-T-Peptide-ACPP-Cysteine:

Peptide sequence:

(SEQ ID NO: 8)
GGGWWSSASPAATLTYTWSGLPAPASPA-eeeeeeee-(Aop)-
PLGC(Me)AG-rrrrrrrrr-GAGAPAC

Lower case indicates D-amino acids
AOP=5-amino-3-oxapentanoyl

### C(Me)=(S-methyl)cysteine

Materials Used

Amino acids:

### Fmoc-Ala-OH

### Fmoc-Pro-OH

### Fmoc-Gly-OH

### Fmoc-Cys (Me)-OH

### Fmoc-Leu-OH

### Fmoc-Aop-OH

### Fmoc-Ser(tbu)-OH

### Fmoc-Trp(Boc)-OH

### Fmoc-Thr(tbu)-OH

### Fmoc-Tyr(tbu)-OH

### Fmoc-D-Arg(pf)-OH

### Fmoc-D-Glu(Otbu)-OH

### Resin: H-Cys-2Cl-Trt-Cl-Resin

Coupling reagent and base: 2-(1H-Benzotriazole-1-yl)-3,3-Tetramethyluronium hexafluorophosphate (HBTU), N-methylmorpholine (NMM)
Solvents: DMF, DCM, methanol, piperidine

Other Reagents used for the Synthesis:

Kaiser test solution:
A: 80% phenol+20% anhydrous ethanol
B: distilled pyridine
C: 5g ninhydrin+100 mlL anhydrous ethanol
Fmoc deprotection solution:
20% piperidine in DMF
The cleavage solution:
87.5% TFA+5% phenyl methyl thioanisole+2.5% phenol+2.5% EDT+2.5% H2O

Procedure for the Synthesis of the Peptide

a. Preparing the resin: The weighed H-Cys-2Cl-Trt-Cl-Resin is allowed to swell in DCM for 30 min and then dried under vacuum.
b. Preparing amino acid and HBTU solution for coupling reaction: dissolve 3 eq of amino acid and 2.85 eq of HBTU in DMF.
c. Coupling reaction: add the solution of the amino acid and HBTU in DMF into the resin and then add 6 eq of N-methylmorpholine (NMM). The resin is agitated using a stream of N2 for 30 min.
d. Washing: remove the reaction solution by suction and wash the resin by DMF (3×2 min).
e. Kaiser test: place small amount of resin after the coupling reaction in a test tube, add the Kaiser test solution (of A, B and C) 2 drops each. Heat the resin/solution at 110° C. for 3 min. If the solution is pale and the resin is clear, the coupling reaction is complete.
f. Fmoc Deprotection: The resin is treated with the deproection solution whilst agitated with N2 stream for 30 min, then washed with DMF (6×2 min), dried under vacuum.
g. The steps b to f are repeated till the last amino acid is attached to the peptide.
h. Final wash of the resin: the resin before cleavage requires extensive washing to remove any reagents from the reactions. The resin is washed with methanol (1×2 min), and then DCM (3×2 min), methanol (2×2 min), and then dried under vacuum for 12 h prior to cleavage.

Cleavage of the Peptide to Produce the Crude Peptide

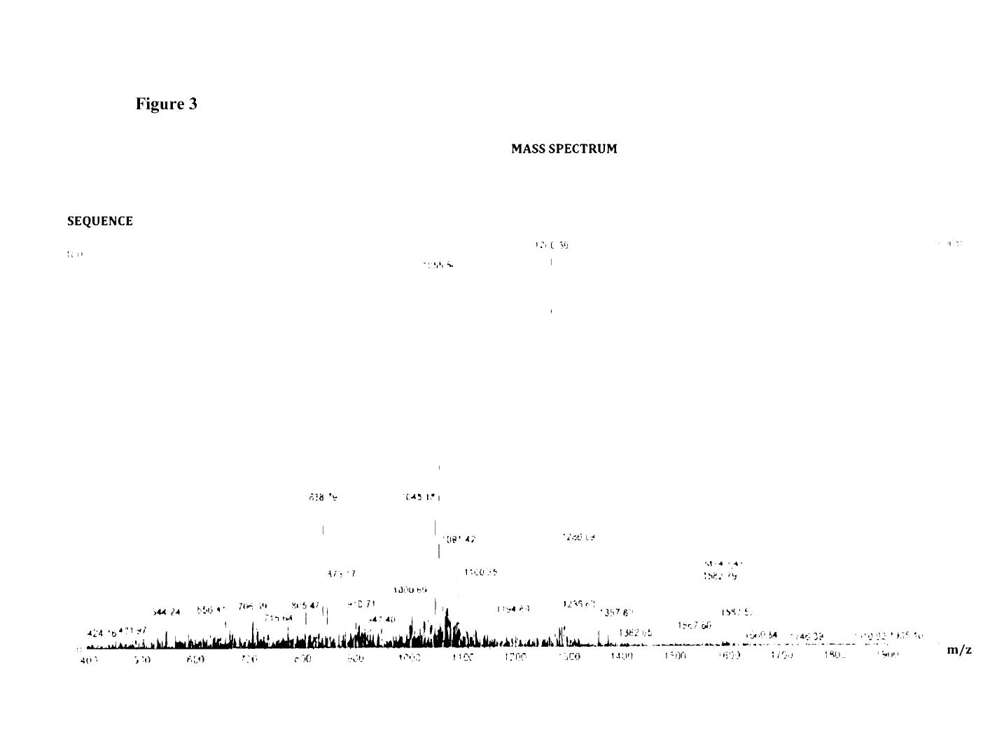

For every 1 g of resin, it requires 10 ml of the cleavage solution. A mixture of the resin and cleavage solution are placed on a shaker at 25° C. for 2 h, The resultant cleavage solution is filtered to remove the resin. To the filtrate (solution) is added 6-8 volume of diethyl ether whilst stirring to principate the crude peptide. The ether mixture is centrifuged and the supernatant is decanted. The peptide is washed with diethyl ether and then the mixture is centrifuged again. The supernatant is discarded. Repeat this washing procedure for 5 times in total to give the crude peptide as white powder. The above washed peptide is dried under vacuum for 24 h. The crude peptide is weighed and then purified by high-performance liquid chromatography (HPLC). The mass spectrum analysis of the GGG-T-peptide-ACPP-cysteine peptide is shown in FIG. 3.

One advantage of using the solid phase peptide synthesis is the precision of the amino acid sequencing. The T-peptide-LPETG is useful for sortase conjugation to fluorescent dyes such as GGG-GFP or GGG-NIR.

### Example 2: Sortase Coupling ©f T-Peptide-LPETG to Fluorescent Labels

Evaluating the binding of T-peptide under fluorescence imaging requires conjugation of the peptide to a fluorescent dye e.g. Green Fluorescent Protein (GFP) for visualization in the 488 nm FITC channel or a near infrared (NIR) dye for 800 nm channel visulatization. This coupling step also tests the feasibility of site specific conjugation to the peptide at its C terminal using sortase reaction.

1. Preparation of Tpeptide-GFP

The sequence of the T-peptide used in this reaction was:

(SEQ ID NO: 9)
H-TLTYTWSGGGLPTGGHHHHHH-OH

N-terminal ‘H’ in the sequence denotes a free amine and C terminal ‘OH’ denotes free acid.

Using a molar ratio of 3GGG:1LPETG:3Sortase for the reaction, 2422 mg of GGG-GFP was reacted with 70 mg of T-peptide-LPETG using 1553 mg of sortase A enzyme in sortase reaction buffer for 5 hours at 37° C. Anti-His-tag nickel coated beads were used for the removal of excess sortase and unbound T-peptide-LPETG (containing a 6His-tag sequence). However, the difficulty was with clearing the excess unbound GGG-GFP because of the small size difference between the T-peptide conjugated GFP and the unbound GGG-GFP.

2. Preparation of T-Peptide-NIR

To attach a near infrared dye to the T-peptide, we used a small commercially synthesized GGG peptide tagged to a near infrared dye(L-Cysteine Dylight 800 C5 Maleimido). The sequence of the GGG-NTR peptide was:

(SEQ ID NO: 10)
H-GGGWWSS-(L-Cysteine Dylight 800 C5 Maleimido)-
OH

N-terminal ‘H’ in the sequence denotes a free amine and C terminal ‘OH’ denotes free acid.

Using a molar ratio of 3GGG:ILPETG:3Sortase for the reaction, 796 mg of GGGNIR was reacted with 350 mg of T-peptide-LPETG using 7769 mg of sortase A enzyme in sortase reaction buffer for 5 hours at 37° C. Anti-His-tag nickel coated beads were used for the removal of excess sortase and unbound T-peptide-LPETG (containing a his-tag sequence). However, the difficulty was with clearing the excess unbound GGG-NIR (mw:1836 daltons) because of the small size difference between it and the T-peptide-LPETG (mw: 2419 daltons).

The conjugation of GFP to the T-peptide is useful for testing the binding of the T-peptide to unstable plaques in vivo because the small peptide of only 2419 daltons, when enhanced by an additional 27,900 daltons in weight by the addition of the GFP, will have a longer circulation time because it will not be rapidly cleared by renal filtration. Conjugating 2 similar sized peptides (T-peptide and GGG-NIR) makes purification by weight differential quite difficult.

### Example 3: Ex Vivo Binding of a T-Peptide-FITC Conjugate to Unstable Mouse Plaque

Conjugates of T-peptide (TLTYTWS)-FITC (Alexa 488) and control peptide (Con-peptide)-FITC were commercially synthesized (GL Biochem, Shanghai China) using solid phase peptide synthesis, as described above. The concentration of the synthesized peptide-FITC conjugates was 1 mg/ml.

T-peptide-FITC conjugate:
(SEQ ID NO: 11)
H-TLTYPATSGK-(FITC)-OH

The control peptide was a scrambled, non-binding peptide comprising the sequence H-GLGYGWSGK(FITC)-OH (SEQ ID NO:12), where the threonine residue of the T-peptide sequence was replaced with a glycine residue.

Creating the TS mouse model of unstable plaques:

At 12 weeks of age and 6 weeks after commencement of high fat diet, ApoE−/− mice (C57BL/6J background) were anaesthetized by intraperitoneal injection of a mixture of ketamine (100 mg/kg) and xylazine (10 mg/kg). An incision was made in the neck and the right common carotid artery exposed by dissecting away circumferential connective tissue. A tandem stenosis with 150 μm outer diameter was introduced with the distal stenosis placed 1 mm from the carotid artery bifurcation and the proximal stenosis 3 mm from the distal stenosis. A 6-0 blue braided polyester fibre suture was tied around the carotid artery and a 150 μm needle to achieve the stenosis diameter of 150 μm and the needle was subsequently removed after the suture was secured. The incision wound was sutured close and the mice resumed their high fat diet. The presence of the tandem stenosis created haemodynamic patterns that, together with the high fat diet, created plaques in the carotid area before the proximal stenosis with histological features of instability similar to human unstable plaques.

Frozen 6 mm tissue sections of tandem stenosis mouse carotid unstable plaques and aortic arch stable plaques on glass slides were thawed for 30 minutes at room temperature. The T-peptide-FITC and Con peptide-FITC conjugates were diluted to a concentration of 0.1 mg/ml. 20 ml of the T-peptide-FITC or the Con peptide-FITC conjugate was added to the frozen unstable carotid plaque sections and to the stable aortic arch sections and the slides were incubated at room temperature in a covered, humidified box containing wet paper towels, for 60 minutes. When incubation was complete, the slides were washed 3 times in PBS, to clear any excess conjugate that may have bound non-specifically to the tissue section. The slides were then examined by fluorescence microscopy (IX81 Olympus microscope) in the FITC 488 nm channel.

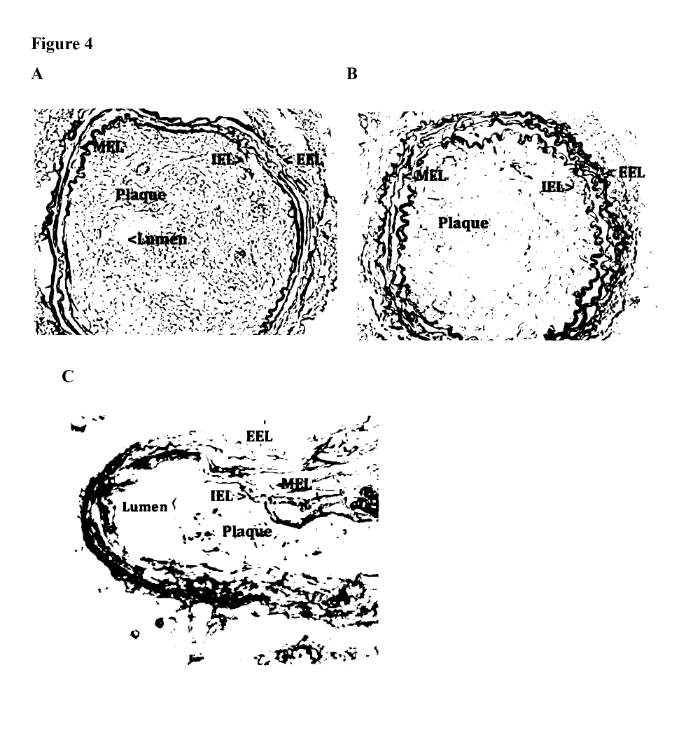

As shown in FIG. 4A, the T-peptide-FITC conjugate bound to an unstable plaque in the mouse carotid artery, as evidenced by a strong fluorescent signal within the lumen. The unstable plaque was large and had almost completely occluded the vascular lumen. By contrast, the control-peptide-FITC conjugate showed no significant binding to a tandem section of the same unstable plaque, although there were signs of autofluorescence along the elastic laminae (FIG. 4B). Similarly, there was no fluorescent signal within the vascular lumen of a stable plaque within the mouse carotid artery, suggesting the absence of binding of the T-peptide to stable aortic arch plaques (FIG. 4C).

These data demonstrate that the T-peptide bound specifically to unstable atherosclerotic plaques.

### Example 4: In Vivo Binding of T-peptide-FITC to Unstable Mouse Plaque

This experiment was undertaken to demonstrate whether the T-peptide is able to enter into and bind specifically to an unstable atherosclerotic plaque in vivo when administered via an intravenous dose to the tandem stenosis mouse.

Method:

The T-peptide-FITC (Alexa 488) and its control scrambled peptide Con-peptide-FITC were commercially synthesized (GL Biochem. Shanghai China) using solid phase peptide synthesis, as described elsewhere herein. The concentration of the synthesized peptides were all at 1 mg/ml,

T-peptide-FITC sequence:
(SEQ ID NO: 11)
H-TLTYTWSGK(FITC)-OH
Control-peptide-HIV sequence:
(SEQ ID NO: 12)
H-GLGYGWSGK-(FITC)-OH

Each mouse with an average weight of 25 g, would receive 100 mg of T-peptide-FITC or 100 mg of Con-peptide-FITC diluted 1:2 to give concentration of 0.5 mg/ml, which works out to be 4 mg peptide per g body weight, 100 ml of the diluted peptide will be given as a bolus into the left external jugular vein and the remaining 100 ml by infusion at a rate of 5 ml per minute using an infusion pump connected to a plastic catheter inserted into the left external jugular vein. The method of bolus dose followed by infusion is to optimize the blood level and duration of the circulating peptide to enhance plaque entry and vascular tissue retention of the peptide, since the small molecular weight of the peptide is at risk of rapid clearance through the kidneys.

At 12 weeks of age, 6 weeks after commencement of High Fat Diet (21% fat, 0.15% cholesterol, Specialty Feeds Pty Ltd, Glen Forrest, Western Australia), ApoE−/− mice (C57/Black6 background) were given a tandem stenosis to their right common carotid artery according to the method used in the paper by Chen et at (2013; Circ Res. 113(3):252-65) After 7 weeks with the tandem stenosis in place, the T-peptide-FITC was administered.

The mice were anaesthetized by intraperitoneal injection of ketamine (100 mg/kg) and xylazine (10 mg/kg) mixture. An incision was made in the neck and the connective tissue dissected to expose the right common carotid artery as well as the left external jugular vein. A 23-G hypodermic needle connected to a plastic catheter was inserted into the left external jugular vein to administer the intravenous peptides. The exposed neck structures were kept moist with a wet gauze. At the end of administration, the mice were kept anesthetized for another 30 minutes before the right common carotid artery segment 1 (unstable plaque) and the aortic arch segment 5 (stable plaque) were dissected out and embedded in OCT cryomatrix for freezing. Cryosections of the frozen plaque tissue of 6 mm thickness were made using the Leica Cryostat (CM 1950, Leica Biosystems Nussloch) and mounted on glass slides. The slides were allowed to dry in room temperature for 30 minutes before storing in −80° C. freezer.

The frozen section slides were thawed for 30 minutes at room temperature before examination under the fluorescence microscope (IX81 Olympus microscope) in the FITC 488 nm channel.

Results

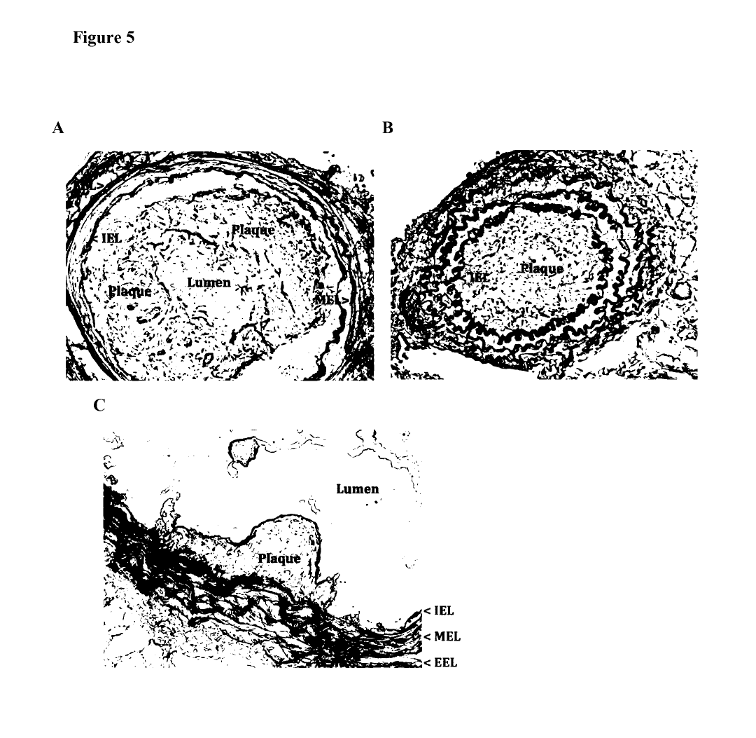

As shown in FIG. 5A, there was strong binding of the T-peptide-FITC conjugate to the unstable plaque following intravenous administration of the T-peptide-FITC conjugate into mice. While there was evidence of autofluorescence from the internal elastic lamina (IEL), middle elastic lamina (MEL) and external elastic lamina (EEL), there was no evidence of binding of the control peptide-FITC conjugate to the unstable plaque following intravenous administration (FIG. 5B). Similarly, there was no binding of the T-peptide-FITC conjugate to the stable aortic arch plaque after intravenous administration (FIG. 5C).

These data show that the T-peptide-FITC conjugate was able to penetrate and specifically bind to unstable atherosclerotic plaques in vivo, whereas no binding was evident in stable plaques present in the segment 5 aortic arch. The elastic laminae of the arterial wall in the mouse arteries showed autofluorescence due to the presence of various elastin fibres that make up the elastic laminae.

### Example 5: In Vitro Binding of T-Peptide-FITC to MMP2 Digested Collagen IV

Method

T-peptide-FITC (Alexa 488) and control peptide-FITC conjugates, as well as the unlabelled T-peptide-LPETG peptide, were commercially synthesized (GL Biochem. Shanghai, China) using solid phase peptide synthesis, as described elsewhere herein. The concentration of the synthesized peptides were all at 1 mg/ml.

T-peptide-FITC sequence:
(SEQ ID NO: 11)
H-TLTYTWSGK(FITC)-OH
Control-peptide-HIV sequence:
(SEQ ID NO: 12)
H-GLGYGWSGK-(FITC)-OH

Binding to MMP2 digested collagen IV
Collagen IV was applied to glass slides coated with polylysine.
1. Polylysine (MW of 30,000-70,000) at 0.1 mg/ml in 0.15 M borate buffer (pH 8.3) was prepared and filtered with 0.22 mm pore size membrane syringe filter (Millex® Syringe Filter, Millipore).
2. Collagen IV (collagen type IV from human fibroblast culture, Sigma. Aldrich) was diluted to 1:10 with Hank's Balanced Salt Solution (HBSS) to 0.04 mg/ml.
3. For MMP2 digestion, Active recombinant human MMP2 enzyme (expressed in CHO cells) was purchased from Calbiochem. MMP2 stock solution was diluted with PBS to create 0.01 μg/μl MMP2 concentration according to manufacturer's recommendations.
4. For T-peptide-HTC testing using added MMP2, 20 μl of polylysine was spotted on several glass slides and left overnight at room temperature to dry. This forms a coating base for collagen to adhere to subsequently.
5. To predigest some collagen IV with MMP2, 3 μl of (0.1 mg/ml ) MMP2 was mixed with 35.8 μl of diluted collagen IV solution with 1.2 μl of 100 mM CaCl2 (3 mM Calcium final concentration) in an Eppendorf tube and incubated at 37° C. shaking at 600 rpm in an Eppendorf Thermomixer for 2 hours.
6. To create intact collagen IV coated slides, 20 ml of diluted collagen IV solution was dropped onto the dried polylysine spot on the slide.
7. To create MMP2 predigested collagen IV coated slides, 20 ml of MMP2 predigested collagen IV was dropped onto the dried polylysine spot on the glass slide.
8. The slides with collagen IV were dried uncovered overnight before testing.
9. Next day, 10 ml of T-peptide-FITC (0.1 mg/ml) was added to the MMP2 predigested collagen spot, 10 ml of T-peptide-FITC (0.1 mg/ml) was added to the intact collagen slide, and 10 ml Control peptide-FITC (0.1mg/ml) was added to the MMP2 predigested collagen slide and the slides were incubated in a covered humidified box with wet paper towels at room temperature for 60 minutes.
10. For the ‘blocked epitope’ slide, 10 ml of unlabelled T-peptide-LPETG (0.1 mg/ml) was added to the MMP2 predigested collagen slide and incubated similarly for 60 minutes. This step binds the epitopes present on the collagen IV and prevents further binding with the FITC labelled T-peptide. Next, 10 ml of T-peptide-FITC (0.1 mg/ml) was added to the same area and incubated for 60 minutes.
11. All the treated slides were examined under fluorescence microscopy (IX81 Olympus microscope).

Results

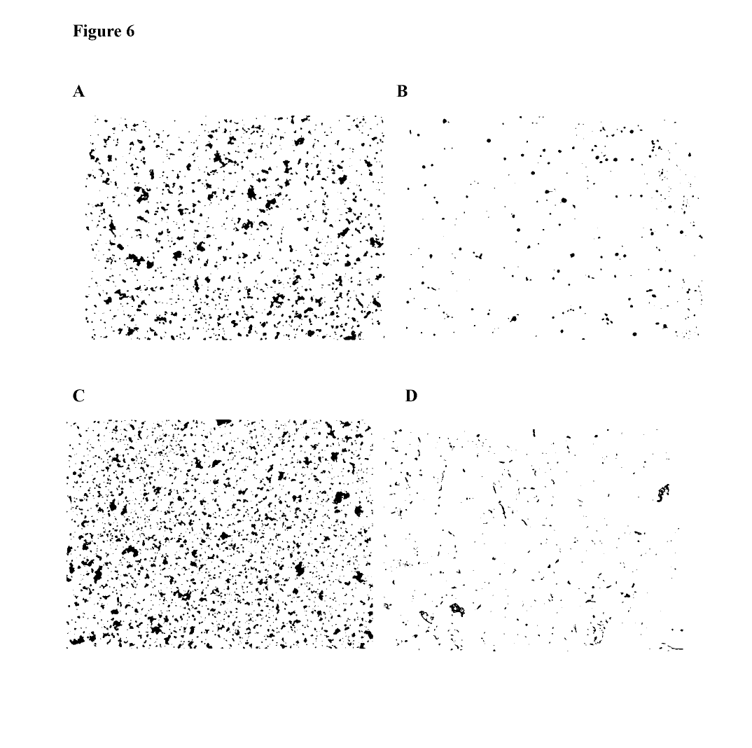

When an MMP2-predigested collagen IV coated glass slide was incubated with the T-peptide-FITC conjugate, there was a strong FITC signal, indicative of binding of the T-peptide to MMP2-predigested collagen IV (FIG. 6A). By contrast, when the T-peptide-FITC conjugate was added to undigested collagen IV coated glass slides, there was no FITC signal, indicative of non-binding of the T-peptide to intact collagen IV fibres (FIG. 6B). Similarly, when the control peptide-FITC conjugate was added to MMP2-predigested collagen IV coated glass slides, there was no FITC signal, indicative of non-binding of the control peptide to MMP2-predigested collagen IV(FIG. 6C). When the T-peptide-FITC conjugate was added to MMP2-predigested collagen IV coated glass slides that had been preincubated with unlabelled T-peptide, there was no significant FITC signal. This suggesting that the unlabelled T-peptide bound to the target epitopes of the collagen IV fibres that were exposed by MMP2-predigestion and subsequently blocked binding by the T-peptide-HTC conjugate.

This study demonstrates that the T-peptide specifically binds to MMP2 digested collagen IV and not to intact collagen IV.

### Example 6: Ex Vivo Binding of T-Peptide-GFP on Human Plaques

Green fluorescent protein (GFP) is a water soluble dye that is not lipophilic. Unlike the near infrared dye (L-Cysteine Dylight 800 C5 Maieimido) which is highly lipophilic and binds non specifically to fatty tissue on plaque tissue during ex vivo experiments, T-peptide-GFP may be more suitable to ex vivo plaque testing.

Method

70 mg of T-peptide-LPETG (SEQ ID NO:15) was conjugated to 2422 mg of GGG-GFP using 1553 mg of sortase enzyme with the sortase reaction molar ratio of 3 GGG-GFP: 1 T-peptide-LPETG: 3 Sortase. The reaction mixture was cleaned up with anti-His-tag nickel coated beads to remove the unbound T-peptide-LPETG and excess sortase enzyme and made up to 500 ml volume with PBS. Excess GGG-GFP, however, could not be removed and would require careful washing steps to clear excess GFP when treating the ex vivo plaque specimens to remove nonspecific background fluorescence. Unreacted GGG-GFP was used as a non-binding control. Assuming that the conjugation yield is 50%, the his-tag clean up of the sortase and unbound T-peptide-LPETG is 100%, the reaction mixture will contain 35 mg of T-peptide conjugated to GFP, with 1614 mg of unbound GGG-GFP.

Frozen 6 mm sections of human carotid endarterectomy plaques on glass slides were thawed for 30 minutes at room temperature. 50 ml of the T-peptide-GFP mixture (containing 3.5 mg of T-peptide that has the GFP tag and 161.4 mg of unbound GFP) was added to the frozen section and incubated at room temperature for 60 minutes. For the control, 161.4 mg of GGG-GFP was mixed in PBS to a volume of 50 ml and this was added to the ‘control’ frozen human carotid plaque section and incubated similarly for 60 minutes. After incubation, the slides were washed in PBS for 3 times, to clear the excess construct and excess unbound GGG-GFP. The slides were then examined by fluorescence microscopy (IX81 Olympus microscope) in the FITC 488 nm channel.

Results:

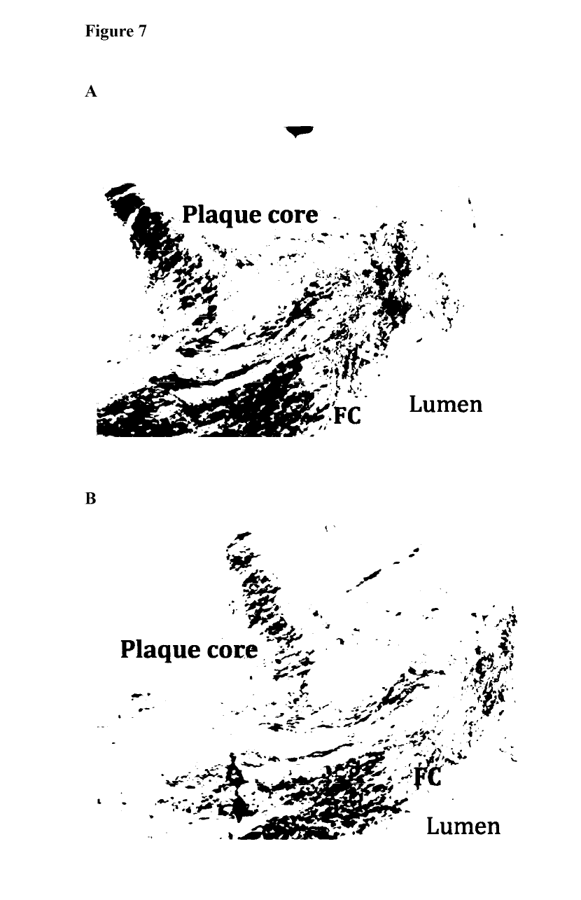

As shown in FIG. 7A, the T-peptide-GFP conjugate bound to parts of the human carotid atherosclerotic plaque, whereas there was no evidence of binding of the control peptide GGG-GFP conjugate on the plaque (FIG. 7B).

This ex vivo experiment demonstrated the presence of MMP2 digested collagen IV on human carotid plaques that possess cryptic binding epitopes for the T-peptide.

### Example 7: Ex Vivo Binding of T-Peptide-FITC to MMP2 Predigested Kidney Sections

This experiment was conducted to determine the specific binding of T-peptide to MMP2-predigested collagen IV in mouse kidney, which comprise an abundance of collagen IV, in particular within the glomeruli.

Method

This study utilizes sortase conjugated T-peptide-GFP as the labelled T-peptide for collagen binding on the kidney sections. The control used was un-reacted GGG-GFP. Using a molar ratio of 3GGG :1LPETG:3Sortase for the reaction, 2422 μg of GGG-GFP was reacted with 70 μg of T-peptide-LPETG using 1553 μg of sortase A enzyme in sortase reaction buffer for 5 hours at 37° C. Anti-His-tag nickel coated beads were used for the removal of excess sortase and unbound T-peptide-LPETG(containing a 6His-tag sequence). However, the difficulty was with clearing the excess unbound GGG-GFP because of the small size difference between the T-peptide conjugated GFP and the unbound GGG-GFP. Assuming that the conjugation yield is 50%, the his-tag clean up of the sortase and unbound T-peptide-LPETG is 100%, the reaction mixture will contain 35 μg of T-peptide conjugated to GFP, with 1614 μg of unbound GGG-GFP.

Normal C57/Black6 mouse kidney frozen sections (6 μm) on slides were thawed for 30 minutes at room temperature. To achieve exogenous MMP2 digestion of the kidney glomerular basement, we used active recombinant human MMP2 enzyme (expressed in CHO cells) purchased from Calbiochem. Diluted the MMP2 stock solution with PBS to create 0.01 μg/μl MMP2 concentration according to manufacturer's recommendations. 4 μl of MMP2 was added to each frozen kidney slide and the slides were placed inside a covered box with wet paper towel to keep the environment moist during incubation and prevent drying out of the slide. These were incubated in a 37° C. oven for 2 hours. The optimal MMP2 amount used was 4 μl as this reduced dislodgement of the kidney tissue after the wash step.

The ‘predigested’ kidney slides were then treated with either 20 μl of T-peptide-GFP mixture (containing 1.4 μg of T-peptide that has the GFP tag and 64.5 μg of unbound GFP) or 64.5 μg of (GGG-GFP in PBS made to a volume of 20 μl as control. The slides were incubated at room temperature for 60 minutes. The slides were flushed with PBS for 3 times to wash away excess or non-specifically adhered construct or GFP. The slides were then examined under fluorescence microscopy in the FITC channel (IX81 Olympus microscope).

MMP2-predigested renal cortex tissue sections showed widespread FITC signal, indicating T-peptide-GFP binding to the renal tissue. By contrast, MMP2-predigested renal cortex tissue sections showed no FITC signal when incubated with control GGG-GFR. Undigested renal cortex tissue sections showed small amounts of FITC signal when incubated with T-peptide-GFP, which suggested there was some binding of the T-peptide to the renal tissue. Similarly, undigested renal cortex tissue sections showed no FITC signal when incubated with control GGG-GFP.

The results demonstrate the specific binding of T-peptide to MMP2-predigested renal cortex. The kidney tissue does contain some endogenous MMP2, which explains why the untreated kidney section showed small amounts of FITC signal when incubated with the T-peptide-GFP.

### Example 8: Ex Vivo Binding of T-Peptide-NIR on Human Carotid Sections

This experiment was conducted to assess the ability of the T-peptide to bind to collagen epitopes that may be present in human carotid plaques.

Method:

Using the sortase reaction, 796 μg of the GGG-NIR (NIR is L-Cysteine Dylight 800 C5 Maleimido) was mixed with 350 μg of T-peptide-LPETG and catalysed with 7769 μg of sortase enzyme in a 1000 μl reaction volume with sortase reaction buffer for 5 hours at 37° C. A His-tag clean-up with anti-His-tag coated nickel beads removes the excess sortase enzyme and the unbound T-peptide. As it is technically challenging to separate out the unbound GGG-NIR from the reaction mixture because of the small size difference between T-peptide-NIR and GGG-NIR of less than 2 kDaltons, the His-tag was used to clean up the reaction mixture without dialysis. An assumption is made that the reaction conjugation rate is 50 percent and that the His-tag clean up removes 50% of the T-peptide (unconjugated) and 100% of the sortase enzyme and leaving 50% of unbound GGG-NIR in the reaction mixture. Based on this assumption, an amount of reaction mixture is assigned for the experiment with an appropriate amount of un-reacted GGG-MR and un-reacted T-peptide for control testing. The assumed concentration is 0.175 μg/μl of T-peptide-NIR and 0.264 μg/μl of unbound GGG-NIR after ⅓ of GGG-NIR was coupled to the T-peptide because the molar ratio was 1LPETG:3GGG for the reaction mixture in order to optimize the rate of conjugation but the actual conjugation is 1 LPETG coupling to 1 GGG.

Human carotid endarterectomy frozen specimens are thawed for 30 minutes to room temperature and placed on glass slides. 6 μl (containing 1.05 μg of T-peptide-NIR and 1.584 μg of unbound GGG-NIR) of the reaction mixture was further diluted with 24 μl PBS to make up 30 μl volume. This is added to the endarterectomy specimen and incubated in the dark at room temperature for 60 minutes. For the negative control, 1.05 μg of unreacted T-peptide-LPETG and 1.584 μg of unreacted GGG-NIR is mixed with PBS to a final volume of 30 μl and added to another endarterectomy section from the same specimen and incubated in the dark for 60 minutes.

After the incubation, PBS is used to wash the specimens thoroughly for 4 times to get rid of any unbound excess GGG-NIR dye.

### Experiment 9: Development of a Peptide-Conjugated Nanosponge Loaded with a Specific MMP14 Inhibitor Drug for Intravenous use that has Specific Horning Ability into Unstable Plaques

The designed construct is a ‘smart’ nano carrier that integrates 3 functional components in order to achieve plaque targeting specificity, environment triggered entry of payload into plaque cells and a sustained drug release mechanism. The T-peptide was utilised as a homing (targeting) agent to bind to MMP-digested collagen IV. This approach imparts specific homing into areas of the plaque with collagen IV that is weakened by MMP2 activity, as illustrated by the data described elsewhere herein.

Material and Methods

All laboratory reagents, chemicals, antibodies and solvents were purchased by Bio-Rad (Hercules, Calif., USA), EMD Millipore Cooperation (Billerica, Mass, USA), Fisher Chemicals (New Jersey, N.J., USA), R&D Systems (Minneapolis, Minn, USA), Invitrogen (Carlsbad, Calif., USA) Merck (New Jersey, N.J., USA), Pierce (Rockford, Ill., USA) and Sigma-Aldrich (St. Louis, Mo., USA) and were used according to manufacturer's instructions. The laboratory consumables were from following commercial sources: Eppendorf (Westbury, N.Y., USA) and BD Bioscience (Bedford, Mass., USA). Buffers and solutions were prepared according to standard protocols.

Detection of Cy 3.5 signals of the MMP14 FRET peptide was performed using the FLUOstar OPTIMA BMG LABTECH 96 microplate fluorescence reader standardized for optic settings of excitation 544 nm and emission 590 nm, temperature settings at 37° C., with readings taken every 10 minutes over a 120 minute duration and double orbital shaking at a width of 4 mm 1 second prior to each cycle. All confocal live cell studies were performed using the Nikon Air inverted microscope (Tokyo, Japan) with live cell imaging chamber (designed by Monash Micro imaging). Tissue sections were obtained by cryo-sectioning frozen tissue using the Leica CM1950 cryostat (Leica Microsystems, Germany).

1. Attachment of Aldehyde Linker to Polyester Nanoparticles

A 1-dram via equipped with stir bar and septum was flamed dried and purged with nitrogen. Polyester nanoparticles (4.6% AVL, 4.9% EVL, 80.0 mg, 5.33×10-7 mol, 1.0 eq) were added to the vial, and the vial was again purged with nitrogen. Particles were dissolved by adding a minimal amount of dimethyl sulfoxide through the septum via syringe. A stock solution of N-succinimidyl-p-formylbenzoate was prepared in DMSO, and the linker (4.0 mg, 1.60×10-5 mol, 30.0 eq) was added through the septum via syringe. The reaction was then allowed to stir at room temperature overnight. The resulting mixture was purified by dialysis against dichloromethane using Snakeskin tubing (10K MWCO) for 24 hours. 1H NMR (400 MHz, d-DMSO) δ: 0.92 (6H, d, CH3), 1.47-1.78 (8H, m, CH2), 1.86 (1H, m, CH), 2.14-2.52 (5H, m, CH2, CH), 3.50-3.65 (14H, m, CH2), 4.08 (4H, m, CH2), 5.04 (2H, m, CH2) 5.73 (1H, m, CH), 7.90-8.10 (4H, m, CH), 10.06 (1H, s, CH) (see FIG. 9).

2. Attachment of T-Peptide to Polyester Nanoparticles

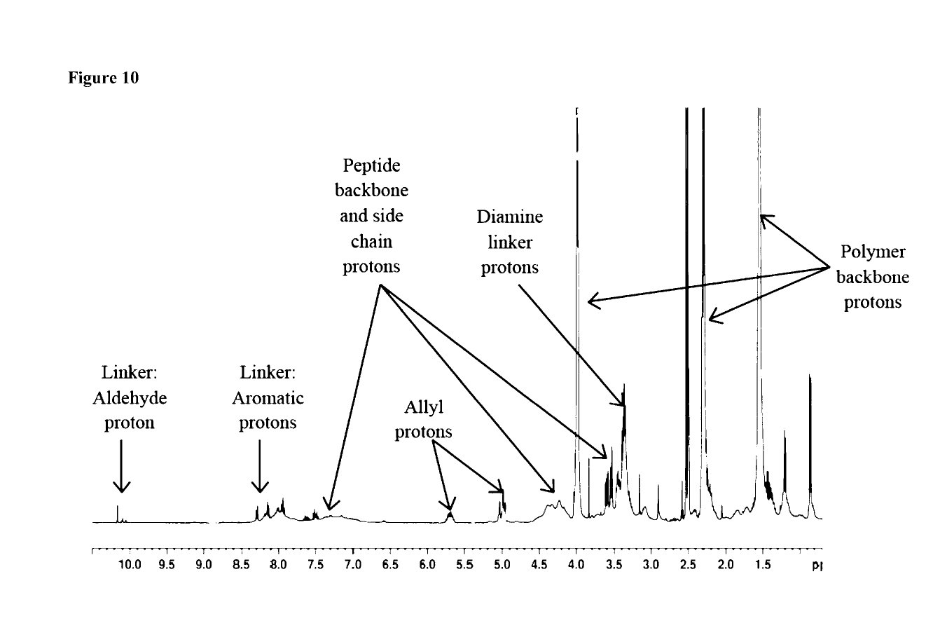

A 1-dram vial equipped with stir bar and septum was flame dried and purged with nitrogen. Polyester nanoparticles (4.6% AVL, 4.9% EVL, 50.0 mg, 3.33×10-7 mol, 1.0 eq) and T-peptide (TLTYTWS; SEQ ID NO:1; 21.2 mg, 3.33×10-6 mol, 0.15 eq/allyl) were added to the vial, and the vial was again purged with nitrogen. Particles and peptide were dissolved by adding d-DMSO through the septum. A stock solution of 2,2-dimethoxy-2-phenylacetophenone was prepared in d-DMSO, and the photoinitiator (1.12 mg, 4.37×10-6 mol, 0.2. eq/allyl) was added to the reaction mixture via syringe. The reaction vial was placed under long wave UV light and allowed to stir at room temperature overnight. Percent attachment of the peptide was calculated using the reduction in the allyl peaks at 5.04 and 5.73 ppm seen in the crude NMR spectrum (see FIG. 10).

3. Attachment of Cy3 Hydrazide Dye to Polyester Nanoparticles

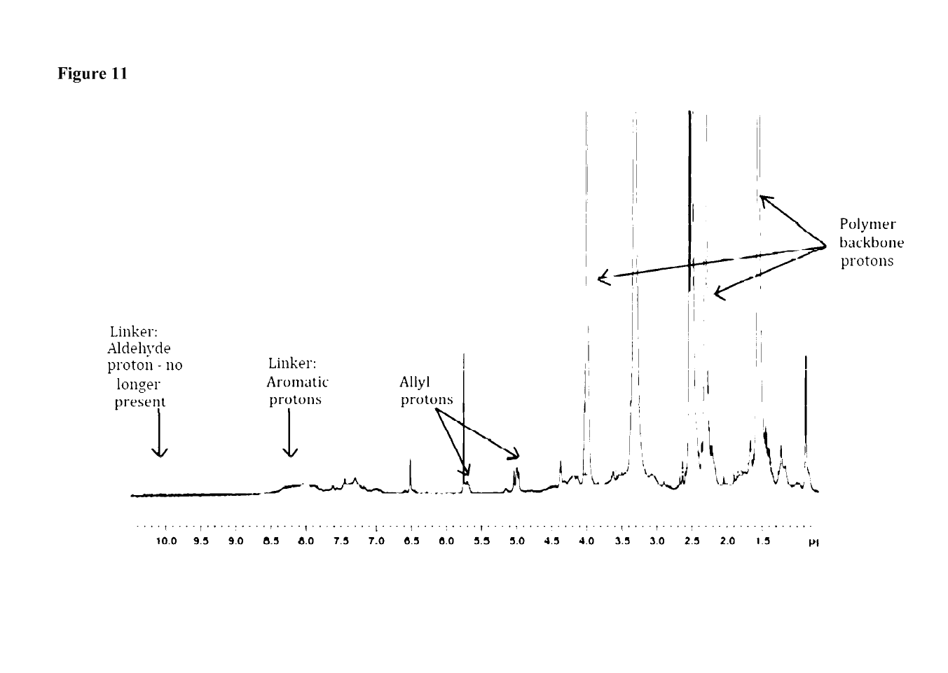

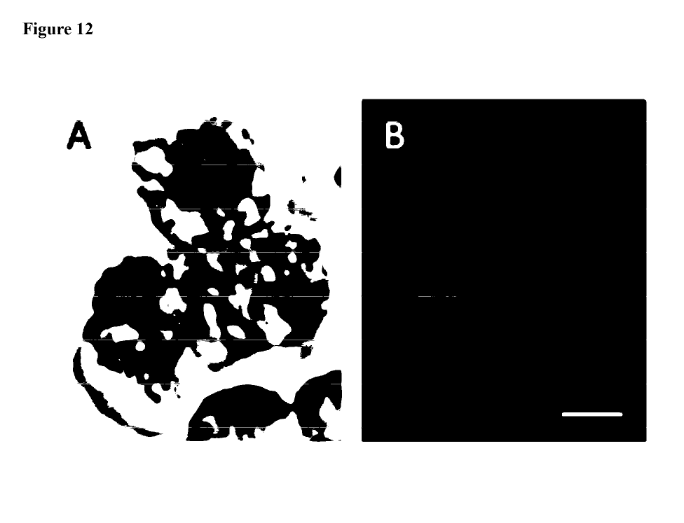

To a 1-dram vial equipped with a stir bar, polyester nanoparticles previously functionalized with an N-succinimidyl-p-formylbenzoate aldehyde linker (50.0 mg, 3.33×10-7 mol, 1.0 eq) were added. Particles not functionalized with T-peptide were added as a dry powder, whereas particles functionalized with T-peptide were transferred back to the original reaction vial from the NMR tube. A stock solution of Cy3 mono hydrazide was prepared in DMSO and the dye (1.58 mg, 2.90×10-6 mol, 8.7 eq) was added to the reaction vial via syringe. The reaction was allowed to stir in the absence of light at room temperature overnight. The resulting mixture was purified by dialysis against a 50:50 v/v mixture of acetonitrile and methanol using Snakeskin tubing (10K MWCO) for 48 hours. Successful attachment of the dye was verified by the absence of the aldehyde peak at 10.06 ppm (see FIG. 11).

4. Loading of Polyester Nanoparticles with Naphthofluorescein

To an Eppendorf tube, D-α-tocopherol polyethylene glycol 1000 succinate (0.0125 mg, 0.59 μL), naphthofluorescein (0.17375 mg, 2.60 μL), and fully functionalized polyester nanoparticles (1.06375 mg, 31.9 μL), all previously dissolved in DMSO, were added. The solution was mixed well until a homogeneous mixture was achieved. Cell culture water (1.00 mL) was added to the tube, and the resulting solution was mixed well. The resulting solution was frozen and then lyophilized to obtain the drug-loaded particles.

5. Confocal Imaging of Live Cells Treated with Targeted Construct

HT1080 murine fibrosarcoma cells and RAW murine macrophage cells were grown on No 1.5 (0.17+/−0.01 mm) Menzel-Glaser cover slips with Dulbecco's Modified Eagle's Medium (DMEM) fortified with 10% fetal calf serum, 1% L-glutamine, 1% non essential amino acids(NEAA) and 1% penicillin/streptomycin cocktail. Cells were incubated with the targeted nanosponge made fluorescent with Cy 3 dye and loaded with Naphthofluorescein drug overnight at 37° C. with 5% CO2. The next day, cells were counter-stained with Cell Tracker Green (Invitrogen) for 30 minutes before confocal imaging. The cell coverslip was mounted into a confocal microscope well containing 500 μl DMEM for live cell imaging. To locate the presence of lysosomes within the RAW murine macrophage cells, we added Lysotracker Blue (Invitrogen) 5 minutes prior to confocal imaging. To determine the nanoparticle localisation and movement, fluorescent and transmitted light images were collected using a Nikon A1r+confocal microscope (Tokyo, Japan); equipped with 60× Water Immersion objective (Nikon 60α Plan Apo VC, WI NA 1,2). Images were collected with the 405, 488 and 568 lasers sequentially to minimise bleed through.

6. In Vivo Testing of Mice with T-Peptide Conjugated to FITC

Mice were anaesthetized by intraperitoneal injection of a mixture of ketamine (100 mg/kg) and xylazine (10 mg/kg). Bolus dose of 50 μl of the FITC labelled collagen homing peptide was given to the tail vein of the test mice followed immediately by a continuous infusion of the FITC peptide at 2.5 μl per minute over 20 minutes and a similar dose of the FITC-labelled scrambled control peptide was given to control mice in the same way. This manner of continuous infusion is to ensure sufficient circulation time for the peptide-FITC conjugate, which has a low molecular weight of less than 10 kDa and may therefore be rapidly cleared by the kidneys. The mice were killed at the end of the 20 minute infusion. After making an incision in the inferior vena cava, each mouse was perfused with PBS through a cardiac puncture with a syringe to purge all blood from the circulation. The right carotid was dissected out and embedded in frozen tissue matrix (OCT), frozen at −80° C. and cryosectioned (Leica Cryostat) into 6 μm thick tissue sections mounted on glass slides. Slides were read under fluorescence microscopy and images were taken at 20× with IX81 Olympus microscope in the FITC channel.

7. In Vivo Testing of Entry of Collagen Peptide Targeted Nanosponge with Cy3

Mice were anaesthetized by intraperitoneal injection of a mixture of ketamine (100 mg/kg) and xylazine (10 mg/kg). Bolus dose of 2.5 mg targeted nanosponges labelled with Cy 3. As the nanosponge size is 150 kDa, which is higher than the renal filtration threshold of 50 kDa, it can be given as a single bolus dose to the tail vein and will remain in circulation for at least several hours. Cy 3 labelled untargeted nanosponge control was given to control mice in the same way. The mice were killed 1 hour after injection. After making an incision in the inferior vena cava, each mouse was perfused with PBS through a cardiac puncture with a syringe to purge all blood from the circulation. The right carotid was dissected out and embedded in frozen tissue matrix (OCT), frozen at −80° C. and cryo-sectioned (Leica Cryostat) into 6 μm thick tissue sections mounted on glass slides. Slides were read under fluorescence microscopy and images were taken at 20× with IX81 Olympus microscope in the TRITC/Cy 3 channel.

8. Assay of MMP14 Activity in Cells Treated with Naphthofluorescein

A peptide sequence that is preferentially cleaved by MMP14 was used to create a FRET probe with N terminal Cy3.5 and C terminal BHQ (Black Hole Quencher): Cy3.5 SGRIGFLRTACBHQ2 (SEQ ID NO:13). The presence of MMP14 activity cleaves this peptide and separates the Cy3.5 from BHQ. HT1080 murine fibrosarcoma cells (natural producers of MMPs) were grown in flasks with Dulbecco's Modified Eagle's Medium (DMEM) fortified with 10% fetal calf serum, 1% L-glutamine, 1% non essential amino acids (NEAA) and 1% penicillin/streptomycin cocktail and 20 μM of naphthofluorescein for 1 week. Control cells were grown similarly but without Naphthofluorescein. After 1 week, the cells were dislodged from the flask without the use of trypsin, by using a cell scraper. This is to prevent trypsin, a broad-spectrum protease, from digesting the MMP14 FRET peptide indiscriminately. The cells were centrifuged at 4000 rcf for 5 minutes, cell pellet was resuspended in PBS containing calcium and magnesium and counted using a haemocytometer. About 50 000 cells were seeded into each test well in a 96 well microplate. Control wells contained PBS with various enzymes added: active MMP2 enzyme, active MMP14, thrombin. The 96 well microplate was incubated at 37° C. inside the FluorSTAR Optima fluorescent microplate reader for 2 hours while Cy 3.5 signal readings in the (Ex544, Em590) channel were taken every 10 minutes. The increase in Cy 3.5 signal from time zero to time 120 minutes is taken as the quantum increase in MMP14 activity on the FRET probe. The readings were plotted as bar charts in Prism software.

9. In Vivo Treatment of Mice with Targeted Drug Construct

Creating the TS mouse model of unstable plaques:

As described elsewhere herein, at 12 weeks of age and 6 weeks after commencement of high fat diet, ApoE−/− mice (C57BL/6J background) were anaesthetized by intraperitoneal injection of a mixture of ketamine (100 mg/kg) and xylazine (10 mg/kg). An incision was made in the neck and the right common carotid artery exposed by dissecting away circumferential connective tissue. A tandem stenosis with 150 μm outer diameter was introduced with the distal stenosis placed 1 mm from the carotid artery bifurcation and the proximal stenosis 3 mm from the distal stenosis. A 6-0 blue braided polyester fibre suture was tied around the carotid artery and a 150 μm needle to achieve the stenosis diameter of 150 μm and the needle was subsequently removed after the suture was secured. The incision wound was sutured close and the mice resumed their high fat diet. The presence of the tandem stenosis created haemodynamic patterns that, together with the high fat diet, created plaques in the carotid area before the proximal stenosis with histological features of instability similar to human unstable plaques.

Testing the in vivo effects of the targeted drug loaded nanosponge on TS mice:

At 5 weeks post tandem stenosis surgery, animals were treated with the targeted drug construct and a series of controls comprising; control PBS, free drug and nondrug loaded nanosponges at 5 weeks after placement of the tandem stenosis, 2 treatment bolus injections were administered via the tail vein 2 weeks apart. Dosages were as follows:

TABLE 1

| Treatment | Targeted | | | Unloaded |
|---|---|---|---|---|
| group | nanosponge | PBS | Free drug | nanosponge |
| Dose per | 2.5 mg construct | 200 μl PBS | 0.35 mg free | 2.15 mg of |
| injection | with 0.35 mg | | naphtho- | empty |
| | naphtho- | | fluorescein | nanosponges |
| | fluorescein | | | |
| | in 200 μl PBS | | | |

Mice were killed 2 weeks after the second dose of treatment. After making an incision in the inferior vena cava, each mouse was perfused with PBS through a cardiac puncture with a syringe to purge all blood from the circulation. The right carotid was dissected out and embedded in frozen tissue matrix (OCT), frozen at −80° C. and cryo-sectioned (Leica Cryostat) into 6 μm thick tissue sections mounted on glass slides.

10. Masson's Trichrome Staining of Mouse Plaque

Frozen slides were thawed to room temperature and then fixed overnight in Bouin's solution and treated with Weighert's solution for 5 minutes, washed under the tap and then stained in Biebrich Scarlet-Acid Fuschin for 15 minutes and then washed. An aqueous solution of Phosphotnolybdic acid hydrate (3%) and Phosphotungstic acid (3% was applied for 15 minutes, followed by Aniline Blue and acetic acid. Stained slides were dehydrated in anhydrous alcohol and cleared in xylene before coverslip mounting in DePex (MERCK, Germany). Images of the Masson's Trichrome slides were acquired using the FSX 100 Olympus microscope system (Olympus, Hamburg, Germany).

11. Haematoxylin and Eosin (H&E) Staining of Mouse Plaque

Frozen slides were thawed to room temperature and then fixed with acetone at −20° C. for 20 min and washed twice for 5 minutes with PBS. They were incubated in Mayer's hematoxylin (Thermo Fisher Scientific Inc., USA) for 6 minutes and washed with distilled water. After that, into Scott's tap water for 30 seconds and washed with distilled water. The sections were then incubated in eosin for 6 minutes, dehydrated 3 times with absolute alcohol for 5 minutes each, followed by twice with Xylene (Recochem™, Quebec, Canada) for 5 minutes each. Finally, the sections were mounted with coverslips using DePex (MERCK, Germany). Images of the H & E slides were acquired using the FSX 100 Olympus microscope system(Olympus, Hamburg, Germany).

12. Picrosirius Red Staining of Mouse Plaque

Frozen slides were thawed to room temperature and submerged in Picrosirius Red (Sirius Red 0.5 g in saturated aqueous picric acid solution 500 ml) solution for 1 hour. Place slides into 0.01 M HCl solution for 2 minutes and rinse under tap water. Dehydrate with anhydrous alcohol and clear with xylene. Finally, the sections were mounted with coverslips using DePex (MERCK, Germany). Images of the Picrosirius Red stained slides were acquired using an Olympus IX81 microscope system (Olympus, Hamburg, Germany) through polarized light. When examined through crossed polarized lenses, birefringence is highly specific for collagen—the larger collagen fibers are bright yellow or orange, and the thinner ones, including reticular fibers, are green.

13. Measurement of Collagen Content of Mouse Plaque using Polasrised Light Microscopy

Collagen quantification of the Picrosirius Red slides were performed using Image J software (National Institute of Health USA). For each mouse, 3 sections of the carotid taken at 30 μm intervals were used to better represent the collagen distribution throughout the length of the plaque. For each section, the quantity of plaque collagen is expressed as the percentage of collagen within the plaque:

$$\frac{Area of collagen in the plaque in the intima layer \times 100}{Total cross sectional area of plaque}$$

The collagen percentages of the 3 sections were then averaged.

14. Statistical Analysis

All quantitative data was analysed by PRISM 6 (GraphPad Software Inc.) and reported as mean +/− one standard deviation. Statistical analysis was performed using ANOVA followed by Tukey's multiple comparison test; with p<0.05 considered statistically significant. Unpaired t test with Welch's correction was also used to analyse the collagen quantification data for the mouse in vivo treatment experiments.

Results:

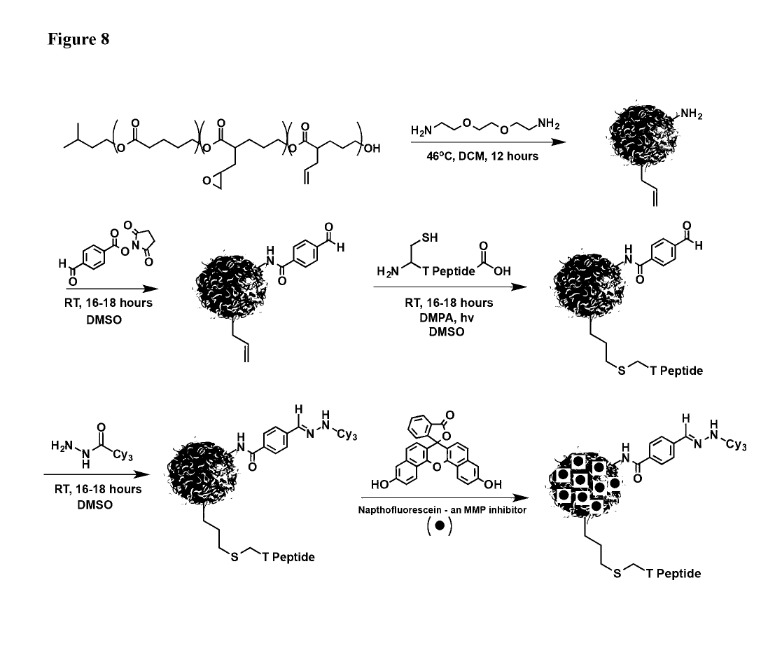

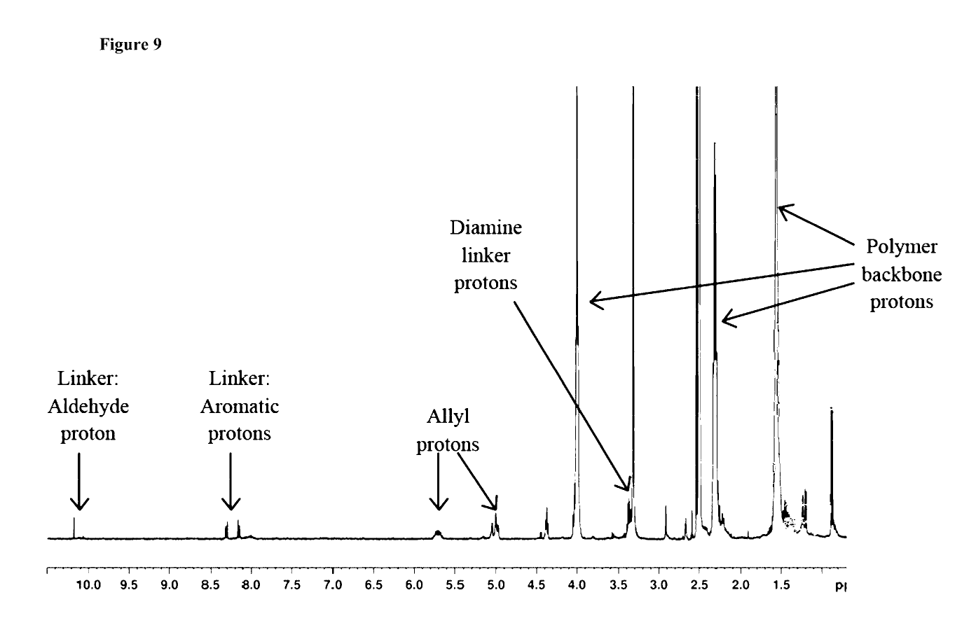

The collagen-homing T-peptide was linked to an activatable cell penetrating peptide (ACPP) that is specifically cleaved by MMP2. The ACPP comprises a polycationic sequence of 9 D-arginine zippered to a polyanionic sequence of 8 D-glutamic acid held together by a U-shaped peptide linker (5-amino-3-oxapentanoyl flexible hydrophilic linker) that contains an amino acid sequence of PLGC(Me)AG (SEQ ID NO:14) that is preferentially cleaved by MMP2. When the ACPP is cleaved at its peptide linker, it releases the polycationic chain of D-amino arginine repeats to penetrate cell membranes by virtue of its net positivity. Any payload attached to this polycationic arm is brought along into cells. A drug carrying polymer nanosponge was attached to the polycationic arm of the ACPP by the thiolene-click reaction (FIG. 8). The mechanism of slow drug release over time is achieved when the polymer disintegrates with hydrolysis. Unlike other MMPs, MMP14 activation is an intracellular process that requires furin activation. In an attempt to spare unrelated MMPs, Naphthofluorescein, a near infrared fluorophore that inhibits furin, was chosen as a drug to specifically inhibit MMP14 activation.

Nanosponges were prepared by first generating linear polymers from polyester precursors using a metal catalyst Sn(OTF)₂ polymerization method (Passarella et al., Cancer Res 2010, 70, 4550). To incorporate aldehyde linkers, polyester nanoparticles were added to a 1-dram vial equipped with stir bar and septum, then purged with nitrogen. Particles were dissolved by adding a minimal amount of dimethyl sulfoxide through the septum via syringe. A stock solution of N-succinimidyl-p-formylbenzoate was prepared in DMSO, and the linker was added through the vial septum via syringe. The reaction was then allowed to stir at room temperature overnight. The resulting mixture was purified by dialysis against dichloromethane using Snakeskin tubing (10K MWCO) for 24 hours. Dynamic light scattering was used to characterize the nanosponges to be about 100 nm in size and static light scattering estimated the molecular weight of the nanosponge as 150 kD. The seven amino acid collagen binding peptide and the ACPP were synthesised by solid phase peptide synthesis and purified using high-performance liquid chromatography (HPLC).

The T-peptide, ACPP and nanoparticles were dissolved by adding d-DMSO. A stock solution of 2,2-dimethoxy-2-phenylacetophenone was prepared in d-DMSO, and the photoinitiator was added to the reaction mixture via syringe. The reaction vial was placed under long wave UV light and allowed to stir at room temperature overnight. Percent attachment of the peptide was calculated using the reduction in the allyl peaks at 5.04 and 5.73 ppm seen in the crude NMR spectrum. Next, polyester nanosponges previously functionalised with an N-succinimidyl-p-formylbenzoate aldehyde linker reacts with a stock solution of Cy3 mono hydrazide prepared in DMSO in the absence of light at room temperature overnight. The resulting mixture was purified by dialysis against a 50:50 v/v mixture of acetonitrile and methanol using Snakeskin tubing (10K MWCO) for 48 hours. Naphthofluorescein, a hydrophobic small molecule drug was loaded as a final step by entrapment into the polyester nanosponges that were fully functionalised with peptide ACPP and Cy3 dye. To an Eppendorf tube, D-α-tocopherol polyethylene glycol 1000 succinate, napthafluorescein, and fully functionalized polyester nanoparticles, all previously dissolved in DMSO, were added. The solution was mixed well until a homogeneous mixture was achieved. Sterile MilliQ water was added to the tube, and the resulting solution was mixed well.

As shown in FIG. 12, effective cell penetration of the ACPP in the presence of active MMP2 was demonstrated using the specific hybridization internalization probe (SHIP) technology (Liu et al.,Angew Chem Int Ed Engl 2013, 52, 5744-5748). An ACPP variant was generated with a C-terminal alkyne functional handle for click chemistry. The SHIP technique uses two 20-mer ssDNA sequences, a fluorescent probe (FIP) with a 5′ fluorophore (Cy5) and a 3′ azide that is “clicked” onto the ACPP using copper click chemistry. The FIP functionalized ACPP was incubated overnight with non-phagocytic saphenous vein endothelial cells (SVEC) overnight. A quencher probe (QPC) comprising a complementary ssDNA sequence with a 3′ Black Hole Quencher 2 (BHQ2) is then added to the cell culture and incubated for 30 minutes at 4° C. to hybridize to any extracellular FIP; quenching its fluorescence, while the fluorescence of the internalized FIP is maintained because the QPC cannot access internalized material. The 4° C. incubation prevents active cell uptake of the QPC.

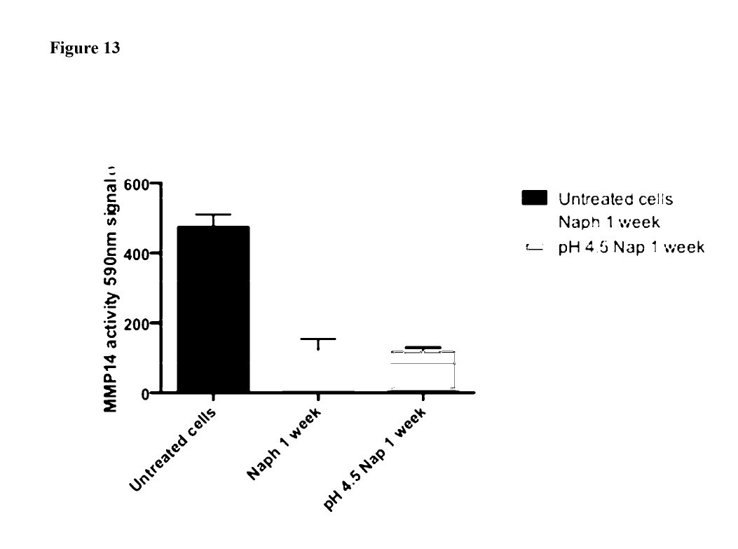

Subsequently, the collagen homing T-peptide, ACPP and nanosponge loaded with Cy3 fluorophore were assembled. Using the murine fibrosarcoma. HT1080 cell line, cellular uptake of the construct was investigated. HT1080 cells naturally produce MMPs including MMP2. These cells were incubated overnight with the T-peptide-ACPP-nanosponge construct at 37° C. The following day, the cell cytoplasm was stained green and the cells were examined by confocal live cell microscopy (Nikon A1) in the FITC and TRITC channels. As shown in FIG. 13, the Cy3 loaded construct was internalised by the cells.

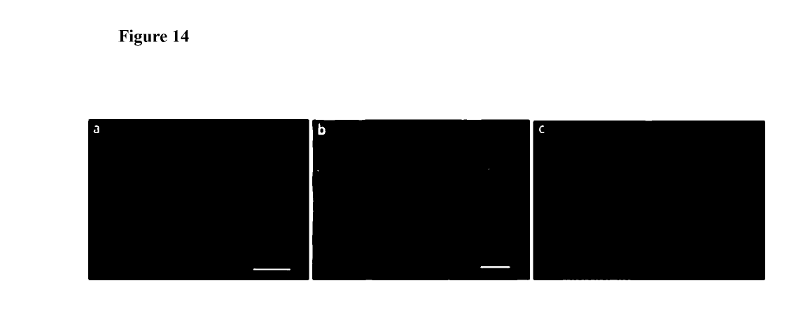

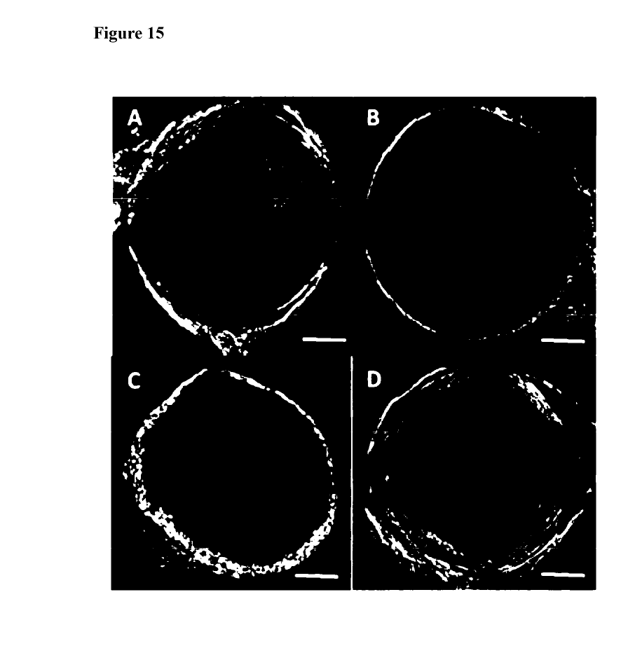

To assess whether the T-peptide-ACPP-nanosponge construct will be effective as a targeted drug system, experiments were conducted to ascertain whether Naphthofluorescein remained efficacious in inhibiting MMP14 activity at pH 5. HT1080 cells were treated with 20 μM of free Naphthofluorescein for 1 week. Another group of HT1080 cells were exposed to Naphthofluorescein that was pre-treated in HCl solution at pH 5 for 30 minutes and then had pH neutralised with NaOH. A FRET peptide was designed with a 5′ Cy3.5 moiety attached to a short amino acid sequence that is preferentially cleavable by membrane type MMPs, such as MMP14, and ends with a 3′ BHQ2 (black hole quencher2). This FRET sequence detects the presence of MMP14 activity as an increase in intensity of Cy3.5 fluorescent signal. The microplate reader scan with the FRET results showed that cells treated with unaltered Naphthofluorescein (Cy3.5 increase: 125.0±17.04, n=3) and pH 5 rendered Naphthofluorescein (126.7±2.728, n=3) both showed significant (p<0.01) suppression of NINTH activity compared to untreated HT1080 cells (473.7±20.93, n=3), indicating that the function of Naphthofluorescein as an MMP14 inhibitor is stable in pH 5 (FIG. 13); We further investigated if the nanoparticle construct entered lysosomes when incubated with murine macrophage RAW cells. Murine RAW cells are naturally phagocytic and they also innately secrete MMPs, including MMP2. By incorporating the ACPP that is cleaved by MMP2, we are in fact creating a nanosponge payload that has a net positive charge from the polycationic D-arginine arm attached to it once the peptide linker of the ACPP is cleaved. This provides a way for ‘lysosomal escape’ of the drug loaded nanosponge by proton sponge effect, allowing the nanosponge to escape back into the cell cytoplasm through the endosomal membrane, avoiding acid degradation of the nanosponge and drug. After an overnight incubation of the Cy3 loaded nanoparticle construct with live RAW cells, the RAW cell cytoplasm was labelled with Cell Tracker Green the next day and 10 minutes before examining the cells under confocal microscopy, we added Lysotracker Blue (Invitrogen) to the RAW cells to visualise lysosomes in the blue DAPI channel. Under confocal microscopy, colocalization of the red and blue fluorescent signals in the RAW cells was assessed, which would indicate the presence of the nanoparticle construct within cell lysosomes (FIG. 14).

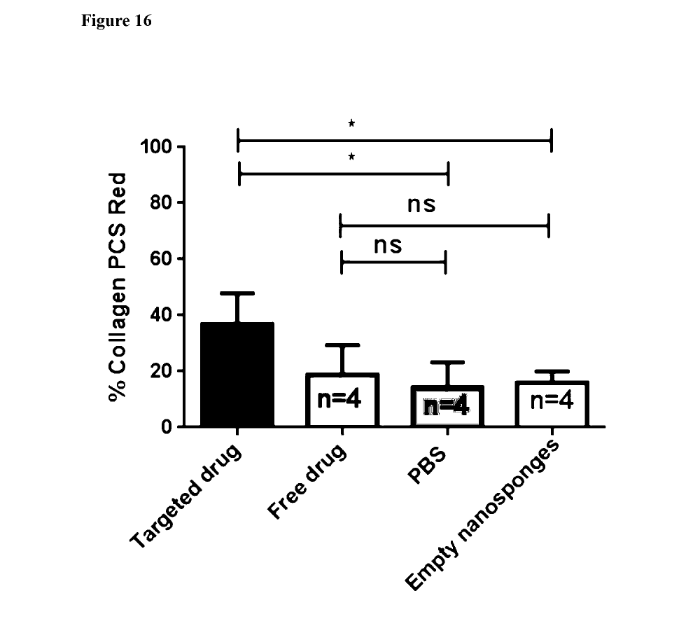

To investigate the effects of Naphthofluorescein on the collagen content of unstable plaques, a novel tandem stenosis mouse model of unstable plaques was utilised. These Apo E knock out mice had sutures in tandem placed at the right carotid artery after 7 weeks of high fat diet to induce formation of unstable plaques. 5 weeks after the tandem stenosis, we gave 2 injections of the Naphthofluorescein loaded targeted nanosponge construct to the mice 2 weeks apart and the same dosing regime was for control mice given PBS, free Naphthofluorescein or drug-free nanosponges. The mice were killed and their right carotid plaques harvested for collagen quantification using Picrosirius Red stain (FIG. 15) Collagen amount, expressed as collagen percentage of the plaque area, revealed marked differences (FIG. 16) between treated (36.88±4.965, n=5) and control mouse groups treated with the free drug (18.87+5.308, n=4), with PBS (14.40±4.522 n=4) and with unloaded empty nanoparticles (16.05±1.994, n=4).

In conclusion, a nanosponge construct was developed that specifically targets unstable plaques with an ability to enhance the collagen content of plaques, thus enhancing their stability and minimizing their risk of rupture. This further illustrates the translational capability of peptide directed nanotechnology in biological applications and that the nanosponge construct represents the potential for the development of biological targeted drug delivery therapeutics in humans for the prevention of conditions associated with rupture of unstable plaques, such as myocardial infarction and strokes.

### Example 10: Ex Vivo Binding of T-Peptide to Mouse Myocardium Post-Myocardial Infarction

The data disclosed elsewhere herein show that the T-peptide allows a clear discrimination between unstable and stable atherosclerotic plaques. To assess whether labelled T-peptide can bind to fibrotic myocardium, a mouse model of myocardial infarction (via left anterior descending coronary artery (LAD) occlusion) was used. This model reflects a very defined area of fibrosis in the infarct zone.

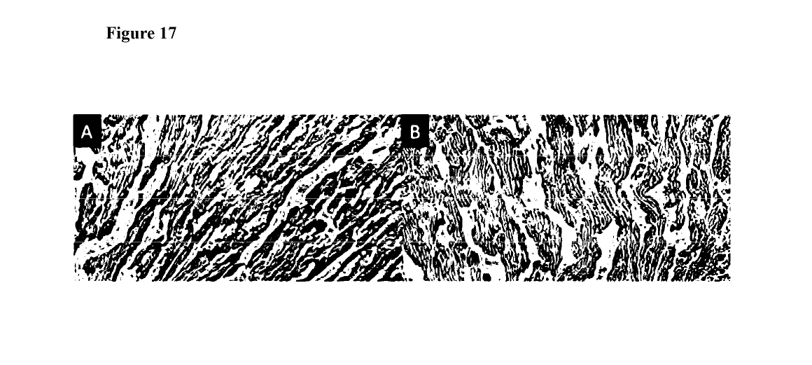

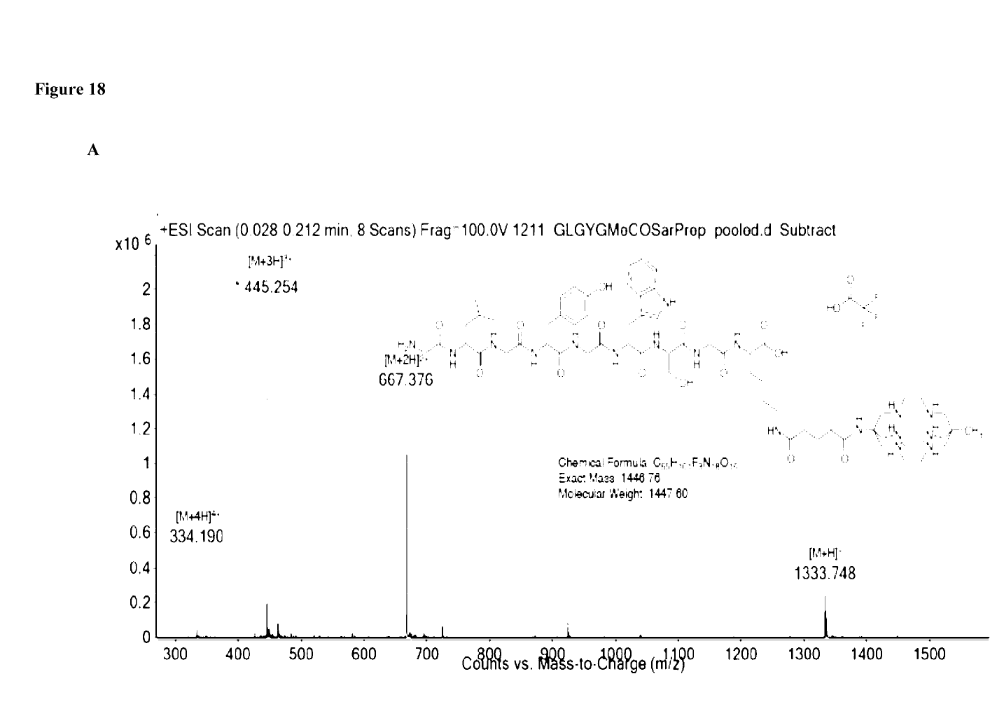

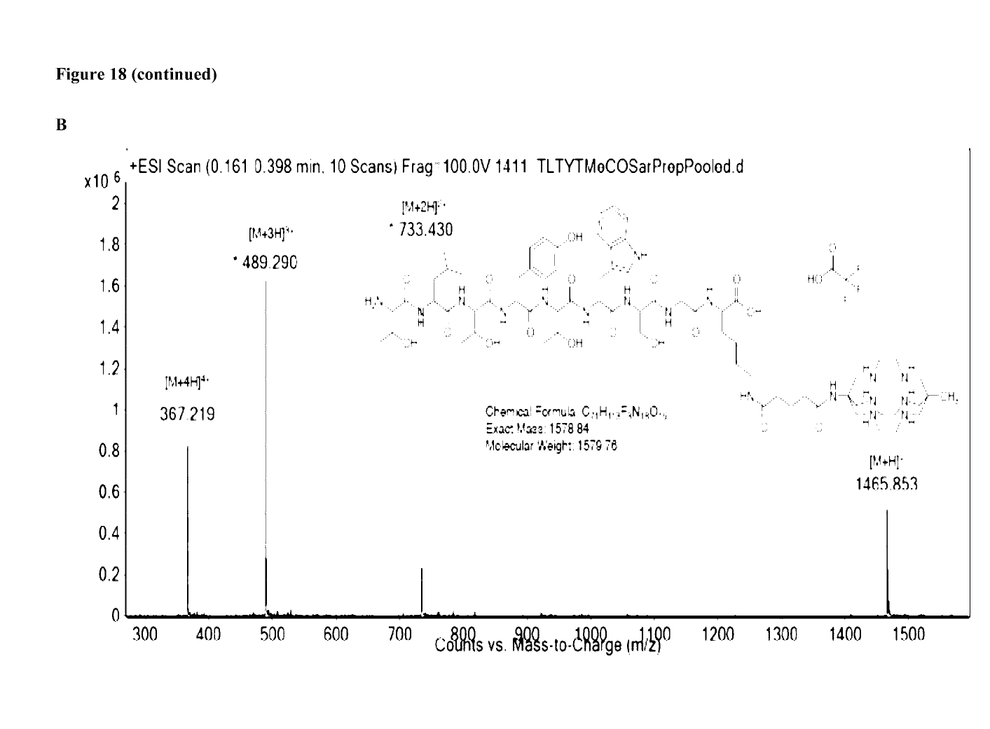

Mice were anaesthetised, the LAD ligated with a suture, and the thorax closed. After 60 mins the suture was removed. After 6 weeks and the development of fibrosis, parts of the infarcted hearts were sectioned and incubated with the T-peptide-FITC and control (s)-peptide-FITC constructs. As shown in FIG. 17, the T-peptide-FITC conjugate binds specifically to areas of localised fibrosis in the myocardium, whereas there was no evidence of binding of the control peptide-FITC conjugate to the same region of fibrotic myocardium.

### Example 11: In Vivo Binding of a Radiolabelled T-Peptide Imaging Agent (Tracer) to Mouse Myocardium Post-Nyocardial Infarction

To confirm these results in a larger animal model, a ⁶⁴Cu-radiolabelled PET tracer (imaging agent) comprising a T-peptide-MeCOSar conjugate was administered to sheep with rapid pacing-induced atrial fibrillation (AF), subsequent myocardial fibrosis and heart failure (HF). The T-peptide-MeCOSar conjugate and the control peptide conjugate (S-peptide-MeCOSar) were prepared using Wang Resin and conjugated using MeCOSar NHS, as follows:

Peptide Synthesis:

Fmoc-Lys(Boc) Wang resin (0.8 mmol/g) was used;
0.25 mmol scale, approx 313 mg resin used in a fritted plastic syringe attached to a vacuum manifold;
Solid phase peptide synthesis (SPPS) conducted:
   Resin was swelled using DCM, washed with DMF;
   Fmoc deprotection conducted using 5% Piperazine in DMF for 30 minutes:
   Resin was washed with 3× DMF, followed by 3× DCM to remove Piperazine;
   Testing for Fmoc deprotection conducted using TNBSA test for detection of free amines (5 μL 10% TNBSA in DMF mixed with 5 μL 10% DIPEA in DMF, added to a few beads of resin in an Eppendorf tube);
   Resin was washed with DMF;
   To a solution of 4 eq Fmoc-AA-OH (amino acid) dissolved in minimal DMF was added 4 eq of HATU, followed by 8 eq of DIPEA;
   Solution was shaken for 5 minutes to allow for activation and then added to the syringe and stirred manually, allowed to react for 30 minutes;
   Washed with 3× DMF, followed by 3× DCM;
   Testing for coupling conducted using TNBSA test for detection of free amines;
Peptide was test cleaved an analyzed by MS to determine completion of synthesis;
Peptide was then cleaved using a solution of 95/2.5/2.5 TFA/H2O/TIPS (20 mL) for 2-3 hours;
Resin was filtered and the filtrate was reduced under a stream of N2 to ˜3 mL;
Solution was diluted with approx. 40 mL Et2O, centrifuged (3 min @ 3k RPM), and the Et2O was decanted. Process was repeated ×3;
Remaining precipitate was dissolved in 50/50 MeCN/H2O and lyophilized.

Peptide Purification (Control):

Initial scouting prep-HPLC run conducted by dissolving crude lyophilized material in 27.5 mL 26% MeCN in 0.1% TFAH2O, approx 7.2 mg/mL, 2.5 mL injected onto Kinetex 5μ C18 100Å AXIA packed prep-HPLC column, 21.2×150 mm, run at 5 mL/min;
Method:
   0-15 min, 20%;
   15-17 min, 20-25%;
   17-72 min, 25-80% (1% /min);
   72-75 min, 80-100%;
   75-95min, 100%;
   Run complete at 96 min;
Fractions containing the control peptide eluted at 42.5-45 min;
Isocratic method developed:
   0-20 min, 28%;
   20-75 min, 35%
Fractions containing the control peptide eluted at 44-65 min;
Approximate yield for control peptide: 45.2 mg, 15% yield.

Peptide Purification (Binding):

Initial scouting prep-HPLC run conducted by dissolving crude lyophilized material in 27.5 mL 38% MeCN in 0.1% TFA/H2O, approx 7.67 mg/mL, 2.8 mL injected onto Kinetex 5μ C18 100Å AXIA packed prep-HPLC column, 21.2×150 mm, run at 5 mL/min;
Method:
   0-15 min, 20%;
   15-17 min, 20-25%;
   17-72 min, 25-80% (1% /min);
   72-75 min, 80-100%;
   75-95 min, 100%;
   Run complete at 96 min;
Fractions containing the control peptide eluted at 41.5-47 min, additional material eluted 50.5-53 min;
Isocratic method developed;
   0-60 min, 36%;
Fractions containing the control peptide eluted at 33-46 min;
Approximate yield for control peptide: 33.2 mg, 9.9% yield;

Conjugation of MeCOSar to Peptides:

To a solution of Fmoc-GLGYGWSGK-OH (17.3 mg) in dry DMF (1 mL) was added MeCOSar-NHS (tris-hydrate/tris-trifluoroacetate) (21.2 mg, 1 eq). The reaction was stirred at 45° C. for 5 hr and monitored by MS, no reaction detected. After 5 hr, NaHCO3 (2 mg) was added to drive the reaction to completion and stirred overnight. Reaction was checked by MS to show complete reaction and removal of N-terminal Fmoc group. The solvent was removed under a stream of N2, dissolved in H2O/MeCN 75/25 with 0.1% TFA, and purified by prep-HPLC.

Yield was approx. 12.5 mg, 62.9%,

To a solution of Fmoc-TLTYTWSGK-OH (17.5 mg) in dry DMF (1 mL) was added MeCOSar-NHS (tris-hydrate/tris-trifluoroacetate) (20.0 mg, 1 eq). The pH of the reaction was adjusted by adding NaHCO3 until reaching pH 9. The reaction was stirred at 45° C. overnight and monitored by MS. After 24 hr, additional NaCO3 was added to drive the reaction to completion. Reaction was checked by MS to show complete reaction and removal of N-terminal Fmoc group. The solvent was removed under a stream of N2, dissolved in H2O/MeCN 75/25 with 0.1% TFA, and purified by prep-HPLC.

Yield was approx. 9.5 mg, 48%.

Loading of the ⁶⁴Cu radiolabel onto the T-peptide-MeCOSar conjugate was prepared by incubating 600 MBq of ⁶⁴Cu was incubated with 1 mL of t-peptide-MeCOSar conjugate (6 nM) in ammonium acetate buffer (0.5 mol/L) at room temperature for 30 min, which typically gives a labelling yield of >98%.

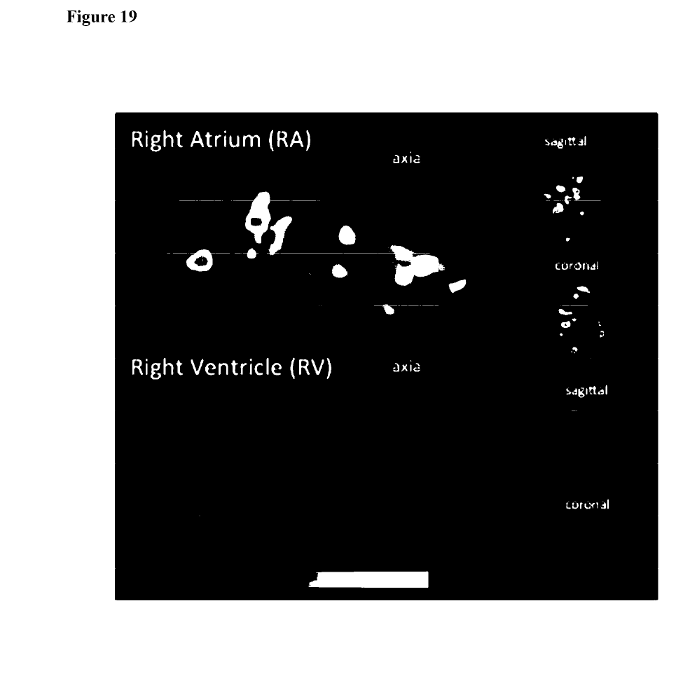

As shown in FIG. 19, the ⁶⁴Cu-radiolabelled T-peptide-MeCOSar PET tracer detected fibrotic tissue in the right atrium of the sheep heart, whereas no binding of the PET tracer was evident in the healthy right ventricle.

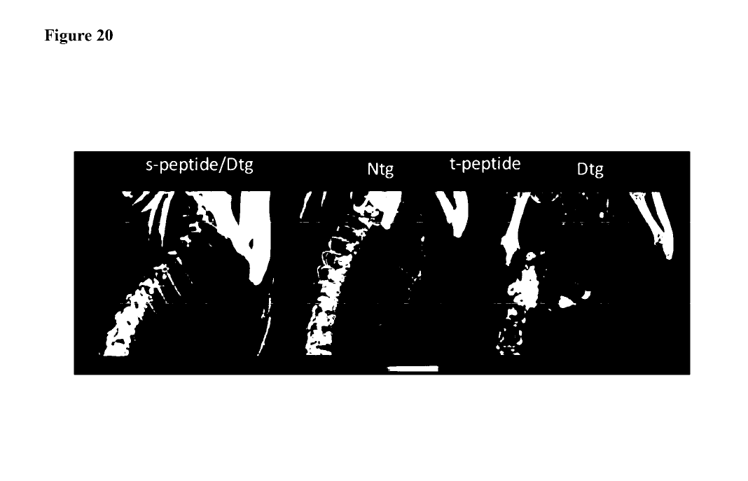

PET/CT scans were also performed in a genetic mouse model of AF, HF and pulmonary fibrosis. Mice were injected with the ⁶⁴Cu-T-peptide-MeCOSar tracer or a non-targeted ⁶⁴Cu-control peptide-MeCOSar via the tail vein. After 30 min, mice were subjected to a Nano-PET/CT scan. An area of the heart and the lungs, which corresponds to the fibrotic tissue, was clearly evident in the PET images (FIG. 20). These findings demonstrate that the imaging agent disclosed herein can be used to detect fibrosis in vivo and guide therapeutic interventions.

1. An imaging agent comprising a polypeptide linked to a detectable label, wherein the polypeptide comprises or consists of the amino acid sequence TLTYTWS (SEQ ID NO:1).

2. (canceled)

3. The imaging agent of claim 1, wherein the detectable label is attached to a complexing agent.

4. The imaging agent of claim 3, wherein the complexing agent is 1,4,7,10-tetraazacyclododecane-1,4,7,10-tetraacetic acid (DOTA) or 5-(8-methyl-3,6,10,13, 16,19-hexaaza-bicyclo[6.6.6]icosan-1-ylamino)-5-oxopentanoic acid (MeCOSar).

5. (canceled)

6. The imaging agent of claim 1, wherein the detectable label is selected from the group consisting of a radio-isotope, an imaging dye, a paramagnetic material and a microbubble.

7. (canceled)

8. The imaging agent of claim 6, wherein the radio-isotope is ⁶⁴Cu.

9. (canceled)

10. A method of detecting fibrosis or an unstable atherosclerotic plaque in a subject in vivo, the method comprising: a) administering to a subject in need thereof the imaging agent of claim 1; and b) detecting the detectable label of the imaging agent, wherein the presence of the detectable label in the subject is indicative of binding of the imaging agent to an area of fibrosis or an unstable atherosclerotic plaque.

11. The method of claim 10, wherein the method for detecting the detectable label is selected from the group consisting of: single photon emission computed tomography; positron emission tomography; near infrared fluorescence imaging; ultrasound imaging; and magnetic resonance imaging.

12-13. (canceled)

14. A therapeutic construct comprising a therapeutic moiety linked to a targeting moiety, wherein the targeting moiety comprises or consists of a polypeptide with the amino acid sequence TLTYTWS (SEQ ID NO:1).

15. (canceled)

16. The therapeutic construct of claim 14, wherein the targeting moiety further comprises a cell penetrating agent.

17. The therapeutic construct of claim 16, wherein the cell penetrating agent comprises a plurality of positively-charged amino acid residues.

18. (canceled)

19. The therapeutic construct of claim 16, wherein the polypeptide is attached to the cell penetrating agent via a linker.

20. The therapeutic construct of claim 19, wherein the linker is specifically cleaved by a matrix metalloproteinase (MMP).

21. (canceled)

22. The therapeutic construct of claim 20, wherein the linker comprises or consists of the amino acid sequence PLGC(Me)AG (SEQ ID NO:14).

23. (canceled)

24. The therapeutic construct of claim 14, wherein the therapeutic moiety comprises a compound capable of inhibiting or activating extracellular matrix turnover.

25. The therapeutic construct of claim 24, wherein the therapeutic moiety comprises a compound capable of inhibiting or activating matrix metalloproteinases (MMP) activity.

26. The therapeutic construct of claim 25, wherein the compound is capable of inhibiting MMP activity.

27. The therapeutic construct of claim 26, wherein the compound is an MMP14 inhibitor.

28. (canceled)

29. The therapeutic construct of claim 14, wherein the therapeutic moiety further comprises a carrier.

30. The therapeutic construct of claim 29, wherein the carrier is a nanoparticle.

31. (canceled)

32. A method of treating or preventing a condition associated with unwanted extracellular matrix turnover in a subject, the method comprising administering to a subject in need thereof the therapeutic construct of claim 14.

33-34. (canceled)


SEQUENCE LISTING (15 sequences)

SEQ ID NO: 1 - protein, 7 aa, Artificial sequence
  Synthetic sequence
TLTYTWS

SEQ ID NO: 2 - protein, 6 aa, Artificial sequence
  Synthetic (modified) sequence
  X (4)..(4): (S-Methyl) Cysteine
PLGXAG

SEQ ID NO: 3 - protein, 12 aa, Artificial sequence
  Synthetic (modified) sequence
  X (12)..(12): alkyne
TLTYTWSACPPX

SEQ ID NO: 4 - protein, 16 aa, Artificial sequence
  Synthetic sequence
GGGTTLTYTWSACPPC

SEQ ID NO: 5 - protein, 24 aa, Artificial sequence
  Synthetic (modified) sequence
  X (24)..(24): hydroxy group (OH)
HTLTYTWSGGGLPETGGHHHHHHX

SEQ ID NO: 6 - protein, 48 aa, Artificial sequence
  Synthetic (modified) sequence
  E (18)..(25): D-amino acids
  X (26)..(26): 5-amino- 3-oxapentanoyl
  X (30)..(30): (S-methyl)cysteine
  R (32)..(40): D-amino acids
  X (48)..(48): L-Propargylglycine
TLTYTWSGLASPAAPAPEEEEEEEEXPLGXAGRRRRRRRRRGAASPAX

SEQ ID NO: 7 - protein, 59 aa, Artificial sequence
  Synthetic (modified) sequence
  E (29)..(36): D-amino acids
  X (30)..(30): 5-amino- 3-oxapentanoyl
  misc_feature (37)..(37): Xaa can be any naturally occurring amino acid
  X (41)..(41): (S-methyl)cysteine
  R (44)..(52): D-amino acids
GGGWWSSASPAATLTYTWSGLPAPASPAEEEEEEEEXPLGXAGRRRRRRRRRGAGAPAC

SEQ ID NO: 8 - protein, 23 aa, Artificial sequence
  Synthetic (modified) sequence
  X (1)..(1): hydrogen
  X (23)..(23): hydroxyl group (OH)
XTLTYTWSGGGLPTGGHHHHHHX

SEQ ID NO: 9 - protein, 10 aa, Artificial sequence
  Synthetic (modified) sequence
  X (1)..(1): hydrogen
  X (9)..(9): (L-Cysteine Dylight 800 C5 Maleimido)
  X (10)..(10): hydroxyl group (OH)
XGGGWWSSXX

SEQ ID NO: 10 - protein, 12 aa, Artificial sequence
  Synthetic (modified) sequence
  X (1)..(1): hydrogen
  X (11)..(11): fluorescein isothiocyanate (FITC)
  X (12)..(12): hydroxyl group (OH)
XTLTYTWSGKXX

SEQ ID NO: 11 - protein, 12 aa, Artificial sequence
  Synthetic (modified) sequence
  X (1)..(1): hydrogen
  X (11)..(11): fluorescein isothiocyanate (FITC)
  X (12)..(12): hydroxyl group (OH)
XGLGYGWSGKXX

SEQ ID NO: 12 - protein, 12 aa, Artificial sequence
  Synthetic (modified) sequence
  X (1)..(1): Cyanine3.5 NHS ester (Cy3.2)
  X (12)..(12): Black Hole Quencher (BHQ)
XGRIGFLRTACX

SEQ ID NO: 13 - protein, 6 aa, Artificial sequence
  Synthetic (modified) sequence
  X (4)..(4): (S-methyl)cysteine
PLGXAG

SEQ ID NO: 14 - protein, 5 aa, Artificial sequence
  Synthetic sequence
LPETG

SEQ ID NO: 15 - protein, 12 aa, Artificial sequence
  Synthetic sequence
TLTYTWSLPETG